(19) United States
(12) Patent Application Publication
(10) Pub. No.: US 2011/0159504 A1
(43) Pub. Date: Jun. 30, 2011

(54) METHYLATION MARKERS FOR SENSITIVITY TO MICROTUBE BASED THERAPIES AND METHODS OF USE

(75) Inventors: James G. Herman, Lutherville, MD (US); Malcolm V. Brock, Owings Mills, MD (US); Johann C. Brandes, Baltimore, MD (US)

(73) Assignee: THE JOHNS HOPKINS UNIVERSITY, Baltimore, MD (US)

(21) Appl. No.: 12/991,647

(22) PCT Filed: May 8, 2009
(86) PCT No.: PCT/US2009/002870

§ 371 Date: Mar. 10, 2011

Related U.S. Application Data
(60) Provisional application No. 61/127,005, filed on May 9, 2008.

(51) Int. Cl. C12Q 1/68

(52) U.S. Cl. 435/6.12

(57) ABSTRACT

The present invention relates to the use of nucleic acid methylation and methylation profiles to detect predict sensitivity of cells to cytotoxic chemotherapies, and in particular to microtubule based therapies, for example taxanes. The invention relates to methods for identifying a methylation profile of the checkpoint with forkhead and ring finger domains gene (CHFR) that is associated with a sensitivity to agents directed at the microtubule.

### RELATED APPLICATIONS

This application claims the benefit of U.S. Provisional Application No. 61/127,005, filed May 9, 2008. The entire contents of the aforementioned application are hereby incorporated herein by reference.

### FIELD OF THE INVENTION

The present invention relates to the use of nucleic acid methylation and methylation profiles to detect predict sensitivity of cells to cytotoxic chemotherapies, and in particular to microtubule based therapies, for example taxanes. The invention relates to methods for identifying a methylation profile of the checkpoint with forkhead and ring finger domains gene (CHFR) that is associated with a sensitivity to agents directed at the microtubule. The method is of particular use as a predictive biomarker for sensitivity to microtubule-based therapy and as a determinant of survival in the treatment of cancer patients with microtubule based agents. The invention further relates to DNA methylation as a predictor of course of treatment, specifically in patients suffering from tumors with CHFR methylation.

### BACKGROUND OF THE INVENTION

DNA methylation is a chemical modification of DNA performed by enzymes called methyltransferases, in which a methyl group (m) is added to certain cytosines (C) of DNA. This non-mutational (epigenetic) process (mC) is a critical factor in gene expression regulation. See, J. G. Herman, Seminars in Cancer Biology, 9: 359-67, 1999. DNA methylation plays an important role in determining gene expression. By turning genes off that are not needed, DNA methylation is an essential control mechanism for the normal development and functioning of organisms. Alternatively, abnormal DNA methylation is one of the mechanisms underlying the changes observed with the development of many cancers. However, a precise role for, and the significance of, abnormal DNA methylation in human tumorigenesis has not been well established.

Loss of gene function is cancer can occur by both genetic and epigenetic mechanisms. The best-defined epigenetic alteration of cancer genes involves DNA methylation of clustered CpG dinucleotides, or CpG islands, in promoter regions associated with the transcriptional inactivation of the affected genes. CpG islands are short sequences rich in the CpG dinucleotide, and can be found in the 5′ region of about half of all human genes. Methylation of cytosine within 5′ CGIs is associated with loss of gene expression and has been seen in a number of physiological conditions, including X chromosome inactivation and genomic imprinting. Aberrant methylation of CpG islands has been detected in genetic diseases such as the fragile-X syndrome, in aging cells and in neoplasia. About half of the tumor suppressor genes which have been shown to be mutated in the germline of patients with familial cancer syndromes have also been shown to be aberrantly methylated in some proportion of sporadic cancers, including Rb, VHL, p16, hMLH1, and BRCA1 (reviewed in Baylin, et al, Adv. Cancer Res. 72:141-196 1998). Methylation of tumor suppressor genes in cancer is usually associated with (1) lack of gene transcription and (2) absence of coding region mutation. Thus CpG island methylation can serve as an alternative mechanism of gene inactivation in cancer.

Although the phenomenon of gene methylation has attracted the attention of cancer researchers for some time, its true role in the progression of human cancers is just now being recognized. In normal cells, methylation occurs predominantly in regions of DNA that have few CG base repeats, while CpG islands, regions of DNA that have long repeats of CG bases, remain non-methylated. Gene promoter regions that control protein expression are often CpG island-rich. Aberrant methylation of these normally non-methylated CpG islands in the promoter region causes transcriptional inactivation or silencing of certain tumor suppressor expression in human cancers.

Genes that are methylated in tumor cells are strongly specific to the tissue of origin of the tumor. Molecular signatures of cancers of all types can be used to improve cancer detection, the assessment of cancer risk and response to therapy. Promoter methylation events provide some of the most promising markers for such purposes.

Cancer treatments, in general, have a higher rate of success if the cancer is diagnosed early, and treatment is started earlier in the disease process. A relationship between improved prognosis and stage of disease at diagnosis can be seen across a majority of cancers. Identification of the earliest changes in cells associated with cancer is thus a major focus in molecular cancer research. Diagnostic approaches based on identification of these changes in specific genes may allow implementation of early detection strategies and novel therapeutic approaches. Targeting these early changes will lead to more effective cancer treatment.

Esophageal cancer is the seventh leading cause of cancer death in the United States. Despite treatment advances particularly in combined modality approaches utilizing chemoradiotherapy and surgery, the overall prognosis, even of localized disease, remains poor. Combination regimens that include the taxane paclitaxel have demonstrated activity, but are not superior and possibly have more toxicity than the standard regimen of cisplatin and 5-fluorouracil (5FU). Integration of molecular markers into clinical decision making as predictors for increased response to specific therapies could improve outcomes. Promoter methylation of the DNA repair gene O-6 Methylguanine Methyltransferase (MGMT) has been identified as a marker for response to alkylating agents in glioma. Loss of MGMT expression through promoter CpG island methylation leads to an inability to repair DNA alkylation damage, tumor cell death, and improved overall patient survival.

The checkpoint with forehead and ring finger domains gene (CHFR), has a mitotic checkpoint function that delays cell cycle entry into metaphase following mitotic stress. CHFR is an ubiquitin-kinase controlling aurora-kinase A and polo-like kinase 1, and excludes cyclin B1 from the nucleus. CHFR expression is frequently lost in cancer by promoter region methylation and less frequently by somatic mutation. CHFR inactivation in cancer cell lines causes an inability to respond to mitotic stress induced by microtubular inhibitors, causing cells to enter metaphase without delay leading to apoptosis. In vitro evidence suggests that CHFR deficient gastric, head and neck, endometrial, and cervical cancer cell lines have increased sensitivity to microtubular inhibitors including taxanes. However, there are no clinical reports of CHFR inactivation for predicting response or survival in patients treated with taxanes.

Accordingly, there is a need in the art for earlier and improved methods of detection of cancer. The ability to predict sensitivity to individual cytotoxic chemotherapies could improve treatment response and survival of cancer patients.

### SUMMARY

The present invention features CHFR methylation as a predictive biomarker for sensitivity to microtubule-directed agents, and as a determinant of survival in treatment of cancer patients, and in particular esophageal cancer patients, with microtubule directed agents, but not other cytotoxic agents. The present invention is based on studies examining CHFR methylation in pre-treatment biopsies from patients treated with taxane or non-taxane-based chemoradiotherapy followed by surgery for locally advanced esophageal cancer. Methylated CHFR in these tumors identified patients with a high pathologic CR (pCR) rate and improved survival following treatment only with taxane-based chemoradiotherapy.

In a first aspect, the invention features methods for identifying a subject that will respond to one or more microtubule-directed therapies by detecting nucleic acid methylation of the checkpoint with forkhead and ring finger domains (CHFR) gene in one or more samples, wherein detecting nucleic acid methylation identifies a subject that will respond to one or more microtubule-directed therapies.

In one embodiment, the subject has been diagnosed with cancer, e.g., esophageal cancer, lung cancer, colon cancer, gastric cancer, or head and neck cancer.

In another embodiment, the one or more microtubule-directed therapies is selected from the group consisting of: taxanes and vinca alkaloids.

In a related embodiment, the methylation is detected in the promoter region.

In another related embodiment, the sample is one or more of blood, blood plasma, serum, cells, a cellular extract, a cellular aspirate, tissues, a tissue sample, or a tissue biopsy. In a related embodiment, the sample is obtained from a subject. In some instances, the subject is suspected of having cancer.

The method of claim 1, wherein the method determines the course of treatment.

In another aspect, the invention provides methods for predicting survival in a subject with cancer comprising: detecting nucleic acid methylation of CHFR in one or more samples, wherein detecting nucleic acid methylation identifies survival in a subject.

In one embodiment, the subject underwent previous treatment with a microtubule-directed therapy.

In another aspect, the invention provides methods for identifying a risk of developing cancer in a subject that was treated with a microtubule-directed agent, comprising: detecting nucleic acid methylation of CHFR in one or more samples, wherein detecting nucleic acid methylation identifies a risk of developing cancer in the subject.

In one embodiment, the one or more microtubule-directed therapies is selected from the group consisting of: taxanes and vinca alkaloids.

In related embodiments, the cancer is esophageal cancer, lung cancer, colon cancer, gastric cancer, or head and neck cancer. In related embodiments, the subject has previously been treated for cancer.

In another related embodiment, the methods of the invention are used to determine a course of treatment for a subject.

In one embodiment, the subject is a human.

In another embodiment, the methods of the invention are performed prior to therapeutic intervention for cancer. In another embodiment, the methods are performed after therapeutic intervention for cancer. In a related embodiment, the therapeutic intervention is microtubule-directed therapy.

In another aspect, the invention provides methods for identifying a subject that will respond to one or more microtubule-directed therapies by extracting nucleic acid from one or more cell or tissue samples, detecting nucleic acid methylation of the CHFR gene in the sample, and identifying the nucleic acid methylation state of the CHFR gene, wherein nucleic acid methylation of CHFR indicates the subject that will respond to one or more microtubule-directed therapies.

In another aspect, the invention provides methods for predicting survival in a subject with cancer by extracting nucleic acid from one or more cell or tissue samples, detecting nucleic acid methylation of the CHFR gene in the sample, and identifying the nucleic acid methylation state of the CHFR gene, wherein nucleic acid methylation of the CHFR gene is indicative of survival in a subject with cancer.

In another aspect, the invention provides methods for identifying a risk of developing cancer in a subject that was treated with a microtubule-directed therapy by extracting nucleic acid from one or more cell or tissue samples, detecting nucleic acid methylation of the CHFR gene in the sample, and identifying the nucleic acid methylation state of the CHFR gene, wherein nucleic acid methylation of the CHFR gene is indicative of a risk of developing cancer in a subject that was treated with a microtubule-directed therapy.

In one embodiment, the subject has been diagnosed with cancer, e.g., esophageal cancer, lung cancer, colon cancer, gastric cancer, or head and neck cancer.

In another embodiment, the one or more microtubule-directed therapies is selected from the group consisting of taxanes and vinca alkaloids.

In another embodiment, the methylation is detected in the promoter region.

In another embodiment, the sample is one or more of blood, blood plasma, serum, cells, a cellular extract, a cellular aspirate, tissues, a tissue sample, or a tissue biopsy. In a related embodiment, the sample is obtained from a subject.

In another embodiment, the methods are used to determine a course of treatment for a subject. In one embodiment, the subject is a human.

In another embodiment, the methods of the invention are performed prior to therapeutic intervention for cancer. In another embodiment, the methods are performed after therapeutic intervention for cancer. In a related embodiment, the therapeutic intervention is microtubule-directed therapy.

In another aspect, the instant invention provides methods of treating a subject having or at risk for having cancer by identifying nucleic acid methylation of the CHFR gene, where nucleic acid methylation indicates having or a risk for cancer; and administering to the subject a therapeutically effective amount of a demethylating agent, thereby treating a subject having or at risk for having cancer.

In one embodiment, the methods are used in combination with one or more microtubule-directed therapies, e.g., taxanes or vinca alkaloids.

In another embodiment, the cancer is esophageal cancer, lung cancer, colon cancer, gastric cancer, and head and neck cancer.

In another embodiment, the methods of the invention further comprise correlating the nucleic acid methylation of the CHFR gene in the sample to the methylation status of the CHFR gene in a control sample. In a related embodiment, the methylation status is compared to a threshold value that distinguishes between individuals with and without cancer.

In another embodiment, the methods of the invention further comprise comparing the nucleic acid methylation of the CHFR gene in the sample with a comparable sample obtained from a normal subject.

In another embodiment, the methods of the invention further comprise detecting nucleic acid methylation of one or more genes wherein the presence of methylation is indicative that the subject will respond to microtubule-directed therapies.

In exemplary embodiments of the invention, the detection of nucleic acid methylation is by a quantitative method. In one embodiment, the detection of nucleic acid methylation is carried out by polymerase chain reaction (PCR) analysis.

In one embodiment, the PCR is methylation specific PCR (MSP). In another embodiment, the methylation specific PCR is multiplex methylation specific PCR.

In another embodiment, the detection of nucleic acid methylation is carried out by a method selected from: bisulfite sequencing, restriction endonuclease treatment and Southern blot analysis.

In another embodiment, the methods of detecting nucleic acid methylation are performed as a high-throughput method.

In another embodiment, the methylation is detected in CpG islands of the CHFR gene. In a related embodiment, the methylation is detected in CpG islands of the promoter region.

In another aspect, the invention provides kits for identifying the nucleic acid methylation state of a CHFR gene comprising gene specific primers for use in polymerase chain reaction (PCR), and instructions for use.

In another aspect, the invention provides kits for detecting cancer by detecting nucleic acid methylation of a CHFR gene, the kit comprising gene specific primers for use in polymerase chain reaction (PCR), and instructions for use.

In exemplary embodiments of the kits, the primers for use in PCR are methylation specific PCR (MSP).

Other aspects of the invention are described infra.

### BRIEF DESCRIPTION OF THE FIGURES

FIG. 1 (A-C) shows methylation of CHFR silences gene expression. A) Methylation-specific PCR (MSP) of the CHFR promoter region was examined in esophageal cancer cell lines. Two cell lines are methylated at this gene promoter region (KYSE150 and KYSE510) while the remaining cell lines are unmethylated. Normal lymphocytes are unmethylated (as are other normal tissue), while the in vitro methylated DNA serves as a positive control for the assay (IVD). B) Expression of CHFR as determined by RT-PCR. The methylated cell lines KYSE150 and 510 lack expression of CHFR at baseline, but re-express the gene at the RNA level after 72 hour treatment with the DNA demethylating agent 5-aza-2′-deoxycytidine (DAC). All other cell lines (unmethylated) express CHFR at baseline and are not affected by treatment with DAC. C) Position of MSP primers and molecular beacon in the promoter region of the CHFR gene.

FIG. 2 is a Table that shows distribution of CHFR methylation according to Taxane treatment. n=81.

FIG. 3 (A and B) shows results of methylation Specific PCR of the CHFR promoter region in esophageal cancer biopsies. A) Representative gels for gel-based MSP and B) real-time PCR curves for Q-MSP are shown. Sample 1568 is an example of an unmethylated cancer, sample 1543 with methylation detected by gel-based MSP was above the cutoff point and sample 1564 has high level methylation.

FIG. 4 is a Table showing Methylation of CHFR determined by gel-based and real-time qMSP.

FIG. 5 is a Table that shows crude and multivariate-adjusted hazard ratios of mortality according to taxane vs. non-taxane based chemotherapy.

FIG. 6 (A-C) shows Kaplan-Meier estimates of overall survival, according to taxane treatment and CHFR Methylation Status. A) Overall survival for esophageal cancer patients that received taxane-based therapy by taxane treatment (N=49). There was no difference between patients exposed to docetaxel vs. paclitaxel in overall survival. B) Overall survival for patients treated with paclitaxel (n=31) was significantly greater when CHFR methylation was present. C) Patients treated with docetaxel (n=18) also had an improvement in survival when CHFR was methylated. Due to the smaller sample size, this finding did not reach statistical significance.

FIG. 7 (A and B) shows Kaplan-Meier estimates of overall survival, according to CHFR promoter methylation status. A) Overall survival of patients treated with a taxane containing combination regimen according to CHFR methylation status (n=49). There was a significantly increased survival favoring CHFR methylation, with a hazard ratio of death for unmethylated CHFR of 3.42; 95 percent confidence interval, 1.42-8.24. B) Overall survival in patients treated with combination regimens that did not contain taxanes did not differ between patients with methylated and unmethylated CHFR (n=32).

FIG. 8 (A-D) shows Kaplan-Meier estimates of overall survival, according to response status. A) Overall survival of the entire cohort (n=81) is significantly higher for patients which achieved a pCR than for those who did not. B) Similar overall survival differences are observed when only patients exposed to taxane chemotherapy are considered C) Overall survival in patients with CHFR methylation according to clinical response (n=18). Patients exposed to taxane chemotherapy who achieve a pCR and with CHFR methylation have a 100% 5-year survival and a significantly superior overall survival over those who did not achieve a pCR. D). Overall survival in patients without CHFR methylation according to clinical response (n=31). For CHFR unmethylated patients treated with taxanes, there was no significant difference in survival regardless of the clinical response

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

By “control” is meant a standard or reference condition.

The phrase “in combination with” is intended to refer to all forms of administration that provide a de-methylating agent, or the methods of the instant invention (e.g. methods of detection of methylation) together with a second agent, such as a chemotherapeutic agent, or a de-methylating agent, where the two are administered concurrently or sequentially in any order.

By “checkpoint with forkhead and ring finger(CHFR)” or CHFR in certain embodiments is meant to refer to the nucleic acid sequence set forth by NCBI accession No. NM_{—}018223 (SEQ ID NO: 1), and the corresponding amino acid sequence set forth NCBI accession No. NP_{—}060693 (SEQ ID NO: 2).

The term “agent” as used herein is meant to refer to a polypeptide, polynucleotide, or fragment, or analog thereof, small molecule, or other biologically active molecule.

As used herein, “methylation” is meant to refer to cytosine methylation at positions C5 or N4 of cytosine, the N6 position of adenine or other types of nucleic acid methylation. Methylation can be detection by, for example, by polymerase chain reaction (PCR), including, but not limited to methylation specific PCR. Portions of the DNA regions described herein will comprise at least one potential methylation site (i.e., a cytosine) and can comprise 2, 3, 4, 5, 6, 7, 8, 9, 10, or more potential methylation sites. In preferred embodiments, methylation is detected using methylation specific polymerase chain reaction (MSP).

As used herein the terms “methylation status” are meant to refer to the presence, absence and/or quantity of methylation at a particular nucleotide, or nucleotides within a portion of DNA. The methylation status of a particular DNA sequence (e.g., a DNA marker or DNA region as described herein) can indicate the methylation state of every base in the sequence or can indicate the methylation state of a subset of the base pairs (e.g., of cytosines or the methylation state of one or more specific restriction enzyme recognition sequences) within the sequence, or can indicate information regarding regional methylation density within the sequence without providing precise information of where in the sequence the methylation occurs. The methylation status can optionally be represented or indicated by a “methylation value.” A methylation value can be generated, for example, by quantifying the amount of intact DNA present following restriction digestion with a methylation dependent restriction enzyme. In this example, if a particular sequence in the DNA is quantified using quantitative PCR, an amount of template DNA approximately equal to a mock treated control indicates the sequence is not highly methylated whereas an amount of template substantially less than occurs in the mock treated sample indicates the presence of methylated DNA at the sequence. Accordingly, a value, i.e., a methylation value, for example from the above described example, represents the methylation status and can thus be used as a quantitative indicator of methylation status. This is of particular use when it is desirable to compare the methylation status of a sequence in a sample to a threshold value. In certain examples, the methylation status is determined for a particular gene, for example a CHFR gene. In preferred embodiments, methylation is detected using methylation specific polymerase chain reaction (MSP).

The phrase “nucleic acid” as used herein refers to an oligonucleotide, nucleotide, polynucleotide, or to a fragment of any of these, to DNA or RNA of genomic or synthetic origin which may be single-stranded or double-stranded and may represent a sense or antisense strand, peptide nucleic acid (PNA), or to any DNA-like or RNA-like material, natural or synthetic in origin. As will be understood by those of skill in the art, when the nucleic acid is RNA, the deoxynucleotides A, G, C, and T are replaced by ribonucleotides A, G, C, and U, respectively.

The term “promoter” or “promoter region” refers to a minimal sequence sufficient to direct transcription or to render promoter-dependent gene expression that is controllable for cell-type specific, tissue-specific, or is inducible by external signals or agents. Promoters may be located in the 5′ or 3′ regions of the gene.

The term “sample” as used herein refers to any biological or chemical mixture for use in the method of the invention. The sample can be a biological sample. The biological samples are generally derived from a patient, preferably as a bodily fluid (such as tumor tissue, lymph node, sputum, blood, bone marrow, cerebrospinal fluid, phlegm, saliva, or urine) or cell lysate. The cell lysate can be prepared from a tissue sample (e.g. a tissue sample obtained by biopsy), for example, a tissue sample (e.g. a tissue sample obtained by biopsy), blood, cerebrospinal fluid, phlegm, saliva, urine, or the sample can be cell lysate. In preferred examples, the sample is one or more of blood, blood plasma, serum, cells, a cellular extract, a cellular aspirate, tissues, a tissue sample, or a tissue biopsy. In preferred embodiments, the sample is from esophageal tumor cells, tissue or origin.

The term “subject” as used herein is meant to include vertebrates, preferably a mammal. Mammals include, but are not limited to, humans.

### DETAILED DESCRIPTION OF THE INVENTION

The invention is based upon the discovery that the methylation of certain genes can serve as a marker for sensitivity of cancer cells to microtubule-directed therapy, and is a prognostic and diagnostic markers in treatment of cancer patients with microtubule based agents, in particular patients with esophageal cancer. DNA methylation of promoter regions leads to gene silencing in many cancers, and here DNA methylation of the checkpoint with forkhead and ring finger domains gene (CHFR) in cancer patients is assessed and can be correlated with clinical outcomes.

### I. Detection of Methylation

DNA methylases transfer methyl groups from the universal methyl donor S-adenosyl methionine to specific sites on the DNA. Several biological functions have been attributed to the methylated bases in DNA. The most established biological function for methylated DNA is the protection of DNA from digestion by cognate restriction enzymes. The restriction modification phenomenon has, so far, been observed only in bacteria. Mammalian cells, however, possess a different methylase that exclusively methylates cytosine residues that are 5′ neighbors of guanine (CpG). This modification of cytosine residues has important regulatory effects on gene expression, especially when involving CpG rich areas, known as CpG islands, located in the promoter regions of many genes.

Methylation has been shown by several lines of evidence to play a role in gene activity, cell differentiation, tumorigenesis, X-chromosome inactivation, genomic imprinting and other major biological processes (Razin, A., H., and Riggs, R. D. eds. in DNA Methylation Biochemistry and Biological Significance, Springer-Verlag, New York, 1984). In eukaryotic cells, methylation of cytosine residues that are immediately 5′ to a guanosine, occurs predominantly in CG poor regions (Bird, A., Nature, 321:209, 1986). In contrast, CpG islands remain unmethylated in normal cells, except during X-chromosome inactivation and parental specific imprinting (Li, et al., Nature, 366:362, 1993) where methylation of 5′ regulatory regions can lead to transcriptional repression. De novo methylation of the Rb gene has been demonstrated in a small fraction of retinoblastomas (Sakai, et al., Am. J. Hum. Genet., 48:880, 1991), and recently, a more detailed analysis of the VHL gene showed aberrant methylation in a subset of sporadic renal cell carcinomas (Herman, et al., Proc. Natl. Acad. Sci., U.S.A., 91:9700, 1994). Expression of a tumor suppressor gene can also be abolished by de novo DNA methylation of a normally unmethylated CpG island (Issa, et al., Nature Genet., 7:536, 1994; Herman, et al., supra; Merlo, et al., Nature Med., 1:686, 1995; Herman, et al., Cancer Res., 56:722, 1996; Graff, et al., Cancer Res., 55:5195, 1995; Herman, et al., Cancer Res., 55:4525, 1995).

In higher order eukaryotes DNA is methylated only at cytosines located 5′ to guanosine in the CpG dinucleotide. This modification has important regulatory effects on gene expression, especially when involving CpG rich areas, known as CpG islands, located in the promoter regions of many genes. While almost all gene-associated islands are protected from methylation on autosomal chromosomes, extensive methylation of CpG islands has been associated with transcriptional inactivation of selected imprinted genes and genes on the inactive X-chromosome of females. Aberrant methylation of normally unmethylated CpG islands has been described as a frequent event in immortalized and transformed cells, and has been associated with transcriptional inactivation of defined tumor suppressor genes in human cancers. Any method that is sufficient to detect methylation is a suitable for use in the methods of the invention. In particular embodiments, PCR analysis is preferred, and more particularly, methylation-specific PCR analysis, for example qualitative methylation specific PCR. Other methods that can be used include, but are not limited to, bisulfate modification to identify changes in DNA methylation of the CHFR gene. This correlates with loss of expression. Additional methods to determine the methylation status of this gene include genomic bisulfite sequencing, MassSPEC methods of methylation detection, and those relying on methylation sensitive restriction digestion of DNA or methylbinding proteins. Any method of determination of DNA methylation at the promoter region of CHFR would be expected to also be able to predict sensitivity to taxane therapies. Other methods which examine loss of expression of the gene, for example RT-PCR approaches, or protein expression, for example immunohistochemistry or western blot analysis, might also be used to determine inactivation of CHFR and thus sensitivity to microtubule-directed agents.

Methylation-sensitive restriction endonucleases can be used to detect methylated CpG dinucleotide motifs. Such endonucleases may either preferentially cleave methylated recognition sites relative to non-methylated recognition sites or preferentially cleave non-methylated relative to methylated recognition sites. Examples of the former are Acc III, Ban I, BstN I, Msp I, and Xma I. Examples of the latter are Acc II, Ava I, BssH II, BstU I, Hpa I, and Not I. Alternatively, chemical reagents can be used which selectively modify either the methylated or non-methylated form of CpG dinucleotide motifs.

Modified products can be detected directly, or after a further reaction which creates products which are easily distinguishable. Means which detect altered size and/or charge can be used to detect modified products, including but not limited to electrophoresis, chromatography, and mass spectrometry. Other means which are reliant on specific sequences can be used, including but not limited to hybridization, amplification, sequencing, and ligase chain reaction, Combinations of such techniques can be uses as is desired. Examples of such chemical reagents for selective modification include hydrazine and bisulfite ions. Hydrazine-modified DNA can be treated with piperidine to cleave it. Bisulfite ion-treated DNA can be treated with alkali.

Other techniques which can be used include technologies suitable for detecting DNA methylation with the use of bisulfite treatment include MSP, Mass Array, MethylLight, QAMA (quantitative analysis of methylated alleles), ERMA (enzymatic regional methylation assay), HeavyMethyl, pyrosequencing technology, MS-SNuPE, Methylquant, oligonucleotide-based microarray.

The ability to monitor the real-time progress of the PCR changes the way one approaches PCR-based quantification of DNA and RNA. Reactions are characterized by the point in time during cycling when amplification of a PCR product is first detected rather than the amount of PCR product accumulated after a fixed number of cycles. The higher the starting copy number of the nucleic acid target, the sooner a significant increase in fluorescence is observed. An amplification plot is the plot of fluorescence signal versus cycle number. In the initial cycles of PCR, there is little change in fluorescence signal. This defines the baseline for the amplification plot. An increase in fluorescence above the baseline indicates the detection of accumulated PCR product. A fixed fluorescence threshold can be set above the baseline. The parameter C_{T} (threshold cycle) is defined as the fractional cycle number at which the fluorescence passes the fixed threshold. For example, the PCR cycle number at which fluorescence reaches a threshold value of 10 times the standard deviation of baseline emission may be used as C_{T} and it is inversely proportional to the starting amount of target cDNA. A plot of the log of initial target copy number for a set of standards versus C_{T} is a straight line. Quantification of the amount of target in unknown samples is accomplished by measuring C_{T} and using the standard curve to determine starting copy number.

The entire process of calculating C_{TS}, preparing a standard curve, and determining starting copy number for unknowns can be performed by software, for example that of the 7700 system or 7900 system of Applied Biosystems. Real-time PCR requires an instrumentation platform that consists of a thermal cycler, computer, optics for fluorescence excitation and emission collection, and data acquisition and analysis software. These machines, available from several manufacturers, differ in sample capacity (some are 96-well standard format, others process fewer samples or require specialized glass capillary tubes), method of excitation (some use lasers, others broad spectrum light sources with tunable filters), and overall sensitivity. There are also platform-specific differences in how the software processes data. Real-time PCR machines are available at core facilities or labs that have the need for high throughput quantitative analysis.

Briefly, in the Q-PCR method the number of target gene copies can be extrapolated from a standard curve equation using the absolute quantitation method. For each gene, cDNA from a positive control is first generated from RNA by the reverse transcription reaction. Using about 1 μl of this cDNA, the gene under investigation is amplified using the primers by means of a standard PCR reaction. The amount of amplicon obtained is then quantified by spectrophotometry and the number of copies calculated on the basis of the molecular weight of each individual gene amplicon. Serial dilutions of this amplicon are tested with the Q-PCR assay to generate the gene specific standard curve. Optimal standard curves are based on PCR amplification efficiency from 90 to 100% (100% meaning that the amount of template is doubled after each cycle), as demonstrated by the slope of the standard curve equation. Linear regression analysis of all standard curves should show a high correlation (R² coefficient.gtoreq.0.98). Genomic DNA can be similarly quantified.

When measuring transcripts of a target gene, the starting material, transcripts of a housekeeping gene are quantified as an endogenous control. Beta-actin is one of the most used nonspecific housekeeping genes. For each experimental sample, the value of both the target and the housekeeping gene are extrapolated from the respective standard curve. The target value is then divided by the endogenous reference value to obtain a normalized target value independent of the amount of starting material.

The above-described quantitative real-time PCR methodology has been adapted to perform quantitative methylation-specific PCR (QM-MSP) by utilizing the external primers pairs in round one (multiplex) PCR and internal primer pairs in round two (real time MSP) PCR. Thus each set of genes has one pair of external primers and two sets of three internal primers/probe (internal sets are specific for unmethylated or methylated DNA). The external primer pairs can co-amplify a cocktail of genes, each pair selectively hybridizing to a member of the panel of genes being investigated using the invention method. The method of methylation-specific PCR (QM-MSP) has been described in US Patent Application 20050239101, incorporated by reference in its entirety herein.

Methylation can be detected using two-stage, or “nested” PCR, for example as described in U.S. Pat. No. 7,214,485, incorporated by reference in its entirety herein. For example, two-stage, or “nested” polymerase chain reaction method is disclosed for detecting methylated DNA sequences at sufficiently high levels of sensitivity to permit cancer screening in biological fluid samples obtained non-invasively.

A method for assessment of the methylation status of any group of CpG sites within a CpG island, independent of the use of methylation-sensitive restriction enzymes, is described in U.S. Pat. No. 6,017,704 incorporated by reference in its entirety herein and described briefly as follows. This method employs primers that specific for the bisulfite reaction such that the PCR reaction itself is used to distinguish between the chemically modified methylated and unmethylated DNA, which adds an improved sensitivity of methylation detection. Unlike previous genomic sequencing methods for methylation identification which utilizes amplification primers which are specifically designed to avoid the CpG sequences, MSP primers themselves are specifically designed to recognize CpG sites to take advantage of the differences in methylation to amplify specific products to be identified by the invention assay. The methods of MSP include modification of DNA by sodium bisulfite or a comparable agent that converts all unmethylated but not methylated cytosines to uracil, and subsequent amplification with primers specific for methylated versus unmethylated DNA. This method of “methylation specific PCR” or MSP, requires only small amounts of DNA, is sensitive to 0.1% of methylated alleles of a given CpG island locus, and can be performed on DNA extracted from paraffin-embedded samples, for example. In addition, MSP eliminates the false positive results inherent to previous PCR-based approaches which relied on differential restriction enzyme cleavage to distinguish methylated from unmethylated DNA.

MSP provides significant advantages over previous PCR and other methods used for assaying methylation. MSP is markedly more sensitive than Southern analyses, facilitating detection of low numbers of methylated alleles and the study of DNA from small samples. MSP allows the study of paraffin-embedded materials, which could not previously be analyzed by Southern analysis. MSP also allows examination of all CpG sites, not just those within sequences recognized by methylation-sensitive restriction enzymes. This markedly increases the number of such sites which can be assessed and will allow rapid, fine mapping of methylation patterns throughout CpG rich regions. MSP also eliminates the frequent false positive results due to partial digestion of methylation-sensitive enzymes inherent in previous PCR methods for detecting methylation. Furthermore, with MSP, simultaneous detection of unmethylated and methylated products in a single sample confirms the integrity of DNA as a template for PCR and allows a semi-quantitative assessment of allele types which correlates with results of Southern analysis. Finally, the ability to validate the amplified product by differential restriction patterns is an additional advantage.

MSP can provide similar information as genomic sequencing, but can be performed with some advantages as follows. MSP is simpler and requires less time than genomic sequencing, with a typical PCR and gel analysis taking 4-6 hours. In contrast, genomic sequencing, amplification, cloning, and subsequent sequencing may take days. MSP also avoids the use of expensive sequencing reagents and the use of radioactivity. Both of these factors make MSP better suited for the analysis of large numbers of samples. The use of PCR as the step to distinguish methylated from unmethylated DNA in MSP allows for significant increase in the sensitivity of methylation detection. For example, if cloning is not used prior to genomic sequencing of the DNA, less than 10% methylated DNA in a background of unmethylated DNA cannot be seen (Myohanen, et al., supra). The use of PCR and cloning does allow sensitive detection of methylation patterns in very small amounts of DNA by genomic sequencing (Frommer, et al., Proc. Natl. Acad. Sci. USA, 89:1827, 1992; Clark, et al., Nucleic Acids Research, 22:2990, 1994). However, this means in practice that it would require sequencing analysis of 10 clones to detect 10% methylation, 100 clones to detect 1% methylation, and to reach the level of sensitivity we have demonstrated with MSP (1:1000), one would have to sequence 1000 individual clones.

“Multiplex methylation-specific PCR” is a unique version of methylation-specific PCR. Methylation-specific PCR is described in U.S. Pat. Nos. 5,786,146; 6,200,756; 6,017,704 and 6,265,171, each of which is incorporated herein by reference in its entirety. Multiplex methylation-specific PCR utilizes MSP primers for a multiplicity of markers, for example three or more different markers, in a two-stage nested PCR amplification reaction. The primers used in the first PCR reaction are selected to amplify a larger portion of the target sequence than the primers of the second PCR reaction. The primers used in the first PCR reaction are referred to herein as “external primers” or DNA primers” and the primers used in the second PCR reaction are referred to herein as “MSP primers.” Two sets of primers (i.e., methylated and unmethylated for each of the markers targeted in the reaction) are used as the MSP primers. In addition in multiplex methylation-specific PCR, as described herein, a small amount (i.e., 1 μl) of a 1:10 to about 10⁶ dilution of the reaction product of the first “external” PCR reaction is used in the second “internal” MSP PCR reaction. The term “primer” as used herein refers to a sequence comprising two or more deoxyribonucleotides or ribonucleotides, preferably more than three, and most preferably more than 8, which sequence is capable of initiating synthesis of a primer extension product, which is substantially complementary to a polymorphic locus strand. Environmental conditions conducive to synthesis include the presence of nucleoside triphosphates and an agent for polymerization, such as DNA polymerase, and a suitable temperature and pH. The primer is preferably single stranded for maximum efficiency in amplification, but may be double stranded. If double stranded, the primer is first treated to separate its strands before being used to prepare extension products. Preferably, the primer is an oligodeoxy ribonucleotide. The primer must be sufficiently long to prime the synthesis of extension products in the presence of the inducing agent for polymerization. The exact length of primer will depend on many factors, including temperature, buffer, and nucleotide composition. The oligonucleotide primer typically contains 12-20 or more nucleotides, although it may contain fewer nucleotides.

Primers of the invention are designed to be “substantially” complementary to each strand of the oligonucleotide to be amplified and include the appropriate G or C nucleotides as discussed above. This means that the primers must be sufficiently complementary to hybridize with their respective strands under conditions that allow the agent for polymerization to perform. In other words, the primers should have sufficient complementarity with a 5′ and 3′ oligonucleotide to hybridize therewith and permit amplification of CpG containing nucleic acid sequence.

Primers of the invention are employed in the amplification process, which is an enzymatic chain reaction that produces exponentially increasing quantities of target locus relative to the number of reaction steps involved (e.g., polymerase chain reaction or PCR). Typically, one primer is complementary to the negative (−) strand of the locus (antisense primer) and the other is complementary to the positive (+) strand (sense primer). Annealing the primers to denatured nucleic acid followed by extension with an enzyme, such as the large fragment of DNA Polymerase I (Klenow) and nucleotides, results in newly synthesized + and − strands containing the target locus sequence. Because these newly synthesized sequences are also templates, repeated cycles of denaturing, primer annealing, and extension results in exponential production of the region (i.e., the target locus sequence) defined by the primer. The product of the chain reaction is a discrete nucleic acid duplex with termini corresponding to the ends of the specific primers employed.

The oligonucleotide primers used in invention methods may be prepared using any suitable method, such as conventional phosphotriester and phosphodiester methods or automated embodiments thereof. In one such automated embodiment, diethylphos-phoramidites are used as starting materials and may be synthesized as described by Beaucage, et al. (Tetrahedron Letters, 22:1859-1862, 1981). One method for synthesizing oligonucleotides on a modified solid support is described in U.S. Pat. No. 4,458,066.

In certain preferred embodiments, methylation of CHFR can be determined by real-time MSP using molecular beacons. The method consists in certain embodiments of using a gene for normalization, e.g. ACTB.

In preferred embodiments, the CHFR promoter sequence detects fully methylated (mCHFR), while ACTB for normalization uses methylation independent primers. Preferable, sequences for forward and reverse primers are as follows, where the CHFR target sequence is on chromosome 12 between positions 131974455 and 131974355, while the ACTB target sequence is on chromosome 7 between positions 5538428 and 5538325, (version 36.1 of the NCBI human genome).

Forward Primer CHFR:
(SEQ ID NO: 3)
5′-GTTATTTTCGTGATTCGTAGGCGAC-3′
Reverse Primer CHFR:
(SEQ ID NO: 4)
5′-CGAAACCGAAAATAACCCGCG-3′
Forward Primer ACTB:
(SEQ ID NO: 5)
5′-TAGGGAGTATATAGGTTGGGGAAGTT-3′
Reverse Primer ACTB:
(SEQ ID NO: 6)
5′-AACACACAATAACAAACACAAATTCAC-3′
Beacon CHFR:
(SEQ ID NO: 7)
5′-CGACATGCGAAGTCGTTTGGTTAGGATTAAAGATGG
ICGAGCGGCATGTCG-3′
Beacon ACTB:
(SEQ ID NO: 8)
5′-CGACTGCGTGIGGGGTGGTGATGGAGGAGGTTTAGG
CAGTCG-3′

The primers used in the invention for amplification of the CpG-containing nucleic acid in the specimen, after bisulfite modification, specifically distinguish between untreated or unmodified DNA, methylated, and non-methylated DNA. MSP primers for the non-methylated DNA preferably have a T in the 3′ CG pair to distinguish it from the C retained in methylated DNA, and the complement is designed for the antisense primer. MSP primers usually contain relatively few Cs or Gs in the sequence since the Cs will be absent in the sense primer and the Gs absent in the antisense primer (C becomes modified to U (uracil) which is amplified as T (thymidine) in the amplification product).

The primers of the invention embrace oligonucleotides of sufficient length and appropriate sequence so as to provide specific initiation of polymerization on a significant number of nucleic acids in the polymorphic locus. Where the nucleic acid sequence of interest contains two strands, it is necessary to separate the strands of the nucleic acid before it can be used as a template for the amplification process. Strand separation can be effected either as a separate step or simultaneously with the synthesis of the primer extension products. This strand separation can be accomplished using various suitable denaturing conditions, including physical, chemical, or enzymatic means, the word “denaturing” includes all such means. One physical method of separating nucleic acid strands involves heating the nucleic acid until it is denatured. Typical heat denaturation may involve temperatures ranging from about 80° to 105° C. for times ranging from about 1 to 10 minutes. Strand separation may also be induced by an enzyme from the class of enzymes known as helicases or by the enzyme RecA, which has helicase activity, and in the presence of riboATP, is known to denature DNA. The reaction conditions suitable for strand separation of nucleic acids with helicases are described by Kuhn Hoffmann-Berling (CSH-Quantitative Biology, 43:63, 1978) and techniques for using RecA are reviewed in C. Radding (Ann. Rev. Genetics, 16:405-437, 1982).

As described herein, any nucleic acid specimen, in purified or nonpurified form, can be utilized as the starting nucleic acid or acids, provided it contains, or is suspected of containing, the specific nucleic acid sequence containing the target locus (e.g., CpG).

When complementary strands of nucleic acid or acids are separated, regardless of whether the nucleic acid was originally double or single stranded, the separated strands are ready to be used as a template for the synthesis of additional nucleic acid strands. This synthesis is performed under conditions allowing hybridization of primers to templates to occur. Generally synthesis occurs in a buffered aqueous solution, preferably at a pH of 7-9, most preferably about 8. Preferably, a molar excess (for genomic nucleic acid, usually about 10⁸: 1 primer:template) of the two oligonucleotide primers is added to the buffer containing the separated template strands. It is understood, however, that the amount of complementary strand may not be known if the process of the invention is used for diagnostic applications, so that the amount of primer relative to the amount of complementary strand cannot be determined with certainty. As a practical matter, however, the amount of primer added will generally be in molar excess over the amount of complementary strand (template) when the sequence to be amplified is contained in a mixture of complicated long-chain nucleic acid strands. A large molar excess is preferred to improve the efficiency of the process.

The deoxyribonucleoside triphosphates dATP, dCTP, dGTP, and dTTP are added to the synthesis mixture, either separately or together with the primers, in adequate amounts and the resulting solution is heated to about 90 C-100 C. from about 1 to 10 minutes, preferably from 1 to 4 minutes. After this heating period, the solution is allowed to cool to room temperature, which is preferable for the primer hybridization. To the cooled mixture is added an appropriate agent for effecting the primer extension reaction (called herein “agent for polymerization”), and the reaction is allowed to occur under conditions known in the art. The agent for polymerization may also be added together with the other reagents if it is heat stable. This synthesis (or amplification) reaction may occur at room temperature up to a temperature above which the agent for polymerization no longer functions. Thus, for example, if DNA polymerase is used as the agent, the temperature is generally no greater than about 40 C. Most conveniently the reaction occurs at room temperature.

In certain preferred embodiments, the agent for polymerization may be any compound or system which will function to accomplish the synthesis of primer extension products, including enzymes. Suitable enzymes for this purpose include, for example, E. coli DNA polymerase I, Klenow fragment of E. coli DNA polymerase I, T4 DNA polymerase, other available DNA polymerases, polymerase muteins, reverse transcriptase, and other enzymes, including heat-stable enzymes (i.e., those enzymes which perform primer extension after being subjected to temperatures sufficiently elevated to cause denaturation). Suitable enzymes will facilitate combination of the nucleotides in the proper manner to form the primer extension products which are complementary to each locus nucleic acid strand. Generally, the synthesis will be initiated at the 3′ end of each primer and proceed in the 5′ direction along the template strand, until synthesis terminates, producing molecules of different lengths. There may be agents for polymerization, however, which initiate synthesis at the 5′ end and proceed in the other direction, using the same process as described above.

Preferably, the method of amplifying is by PCR, as described herein and as is commonly used by those of ordinary skill in the art. Alternative methods of amplification have been described and can also be employed as long as the methylated and non-methylated loci amplified by PCR using the primers of the invention is similarly amplified by the alternative means.

The amplified products are preferably identified as methylated or non-methylated by sequencing. Sequences amplified by the methods of the invention can be further evaluated, detected, cloned, sequenced, and the like, either in solution or after binding to a solid support, by any method usually applied to the detection of a specific DNA sequence such as PCR, oligomer restriction (39), allele-specific oligonucleotide (ASO) probe analysis (40), oligonucleotide ligation assays (OLAs) (41), and the like. Molecular techniques for DNA analysis have been reviewed (42).

Optionally, the methylation pattern of the nucleic acid can be confirmed by restriction enzyme digestion and Southern blot analysis. Examples of methylation sensitive restriction endonucleases which can be used to detect 5′CpG methylation include SmaI, SacII, EagI, MspI, HpaII, BstUI and BssHII, for example.

The present invention provides methods for detecting cells, preferably cancer cells, with DNA methylation in the CHFR gene.

One may use MALDI mass spectrometry in combination with a methylation detection assay to observe the size of a nucleic acid product. The principle behind mass spectrometry is the ionizing of nucleic acids and separating them according to their mass to charge ratio. Similar to electrophoresis, one can use mass spectrometry to detect a specific nucleic acid that was created in an experiment to determine methylation. See Tost, J. et al. Analysis and accurate quantification of CpG methylation by MALDI mass spectrometry. Nuc Acid Res, 2003, 31, 9

One form of chromatography, high performance liquid chromatography, is used to separate components of a mixture based on a variety of chemical interactions between a substance being analyzed and a chromatography column. DNA is first treated with sodium bisulfite, which converts an unmethylated cytosine to uracil, while methylated cytosine residues remain unaffected. One may amplify the region containing potential methylation sites via PCR and separate the products via denaturing high performance liquid chromatography (DHPLC). DHPLC has the resolution capabilities to distinguish between methylated (containing cytosine) and unmethylated (containing uracil) DNA sequences. See Deng, D. et al. Simultaneous detection of CpG methylation and single nucleotide polymorphism by denaturing high performance liquid chromatography. 2002 Nuc Acid Res, 30, 3.

Hybridization is a technique for detecting specific nucleic acid sequences that is based on the annealing of two complementary nucleic acid strands to form a double-stranded molecule. In nucleic acid hybridization reactions, the conditions used to achieve a particular level of stringency will vary, depending on the nature of the nucleic acids being hybridized. For example, the length, degree of complementarity, nucleotide sequence composition (e.g., GC v. AT content), and nucleic acid type (e.g., RNA v. DNA) of the hybridizing regions of the nucleic acids can be considered in selecting hybridization conditions. An additional consideration is whether one of the nucleic acids is immobilized, for example, on a filter.

An example of progressively higher stringency conditions is as follows: 2.times.SSC/0.1% SDS at about room temperature (hybridization conditions); 0.2.times.SSC/0.1% SDS at about room temperature (low stringency conditions); 0.2.times.SSC/0.1% SDS at about 42° C. (moderate stringency conditions); and 0.1.times.SSC at about 68° C. (high stringency conditions). Washing can be carried out using only one of these conditions, e.g., high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically.

One example of the use of hybridization is a microarray assay to determine the methylation status of DNA. After sodium bisulfite treatment of DNA, which converts an unmethylated cytosine to uracil while methylated cytosine residues remain unaffected, oligonucleotides complementary to potential methylation sites can hybridize to the bisulfite-treated DNA. The oligonucleotides are designed to be complimentary to either sequence containing uracil or sequence containing cytosine, representing unmethylated and methylated DNA, respectively. Computer-based microarray technology can determine which oligonucleotides hybridize with the DNA sequence and one can deduce the methylation status of the DNA.

An additional method of determining the results after sodium bisulfite treatment would be to sequence the DNA to directly observe any bisulfite-modifications. Pyrosequencing technology is a method of sequencing-by-synthesis in real time. It is based on an indirect bioluminometric assay of the pyrophosphate (PPi) that is released from each deoxynucleotide (dNTP) upon DNA-chain elongation. This method presents a DNA template-primer complex with a dNTP in the presence of an exonuclease-deficient Klenow DNA polymerase. The four nucleotides are sequentially added to the reaction mix in a predetermined order. If the nucleotide is complementary to the template base and thus incorporated, PPi is released. The PPi and other reagents are used as a substrate in a luciferase reaction producing visible light that is detected by either a luminometer or a charge-coupled device. The light produced is proportional to the number of nucleotides added to the DNA primer and results in a peak indicating the number and type of nucleotide present in the form of a program. Pyrosequencing can exploit the sequence differences that arise following sodium bisulfite-conversion of DNA.

A variety of amplification techniques may be used in a reaction for creating distinguishable products. Some of these techniques employ PCR. Other suitable amplification methods include the ligase chain reaction (LCR (Barringer et al, 1990), transcription amplification (Kwoh et al. 1989; WO88/10315), selective amplification of target polynucleotide sequences (U.S. Pat. No. 6,410,276), consensus sequence primed polymerase chain reaction (U.S. Pat. No. 4,437,975), arbitrarily primed polymerase chain reaction (WO90/06995), nucleic acid based sequence amplification (NASBA) (U.S. Pat. Nos. 5,409,818; 5,554,517; 6,063,603), nick displacement amplification (WO2004/067726).

Sequence variation that reflects the methylation status at CpG dinucleotides in the original genomic DNA offers two approaches to PCR primer design. In the first approach, the primers do not themselves “cover” or hybridize to any potential sites of DNA methylation; sequence variation at sites of differential methylation are located between the two primers. Such primers are used in bisulphite genomic sequencing, COBRA, Ms-SNuPE. In the second approach, the primers are designed to anneal specifically with either the methylated or unmethylated version of the converted sequence. If there is a sufficient region of complementarity, e.g., 12, 15, 18, or 20 nucleotides, to the target, then the primer may also contain additional nucleotide residues that do not interfere with hybridization but may be useful for other manipulations. Exemplary of such other residues may be sites for restriction endonuclease cleavage, for ligand binding or for factor binding or linkers or repeats. The oligonucleotide primers may or may not be such that they are specific for modified methylated residues.

One way to distinguish between modified and unmodified DNA is to hybridize oligonucleotide primers which specifically bind to one form or the other of the DNA. After hybridization, an amplification reaction can be performed and amplification products assayed. The presence of an amplification product indicates that a sample hybridized to the primer. The specificity of the primer indicates whether the DNA had been modified or not, which in turn indicates whether the DNA had been methylated or not. For example, bisulfite ions modify non-methylated cytosine bases, changing them to uracil bases. Uracil bases hybridize to adenine bases under hybridization conditions. Thus an oligonucleotide primer which comprises adenine bases in place of guanine bases would hybridize to the bisulfite-modified DNA, whereas an oligonucleotide primer containing the guanine bases would hybridize to the non-modified (methylated) cytosine residues in the DNA. Amplification using a DNA polymerase and a second primer yield amplification products which can be readily observed. Such a method is termed MSP (Methylation Specific PCR; U.S. Pat. Nos. 5,786,146; 6,017,704; 6,200,756). The amplification products can be optionally hybridized to specific oligonucleotide probes which may also be specific for certain products. Alternatively, oligonucleotide probes can be used which will hybridize to amplification products from both modified and nonmodified DNA.

Another way to distinguish between modified and nonmodified DNA is to use oligonucleotide probes which may also be specific for certain products. Such probes can be hybridized directly to modified DNA or to amplification products of modified DNA. Oligonucleotide probes can be labeled using any detection system known in the art. These include but are not limited to fluorescent moieties, radioisotope labeled moieties, bioluminescent moieties, luminescent moieties, chemiluminescent moieties, enzymes, substrates, receptors, or ligands.

Still another way for the identification of methylated CpG dinucleotides utilizes the ability of the MBD domain of the McCP2 protein to selectively bind to methylated DNA sequences (Cross et al, 1994; Shiraishi et al, 1999). Restriction enconuclease digested genomic DNA is loaded onto expressed His-tagged methyl-CpG binding domain that is immobilized to a solid matrix and used for preparative column chromatography to isolate highly methylated DNA sequences.

Real time chemistry allows for the detection of PCR amplification during the early phases of the reactions, and makes quantitation of DNA and RNA easier and more precise. A few variations of the real-time PCR are known. They include the TAQMAN system and MOLECULAR BEACON. system which have separate probes labeled with a fluorophore and a fluorescence quencher. In the SCORPION system the labeled probe in the form of a hairpin structure is linked to the primer. DNA methylation analysis has been performed successfully with a number of techniques which include the MALDI-TOFF, MassARRAY, MethyLight, Quantitative analysis of ethylated alleles (QAMA), enzymatic regional methylation assay (ERMA), HeavyMethyl, QBSUPT, MS-SNuPE, MethylQuant, Quantitative PCR sequencing, and Oligonucleotide-based microarray systems.

The number of genes whose silencing is tested and/or detected can vary: one, two, three, four, five, or more genes can be tested and/or detected. In some examples, methylation of at least one gene is detected. In other examples, methylation of at least two genes is detected. However, methylation of any number of genes may be detected, using the methods as described herein.

For purposes of the invention, an antibody or nucleic acid probe specific for a gene or gene product may be used to detect the presence of methylation either by detecting the level of polypeptide (using antibody) or methylation of the polynucleotide (using nucleic acid probe) in biological fluids or tissues. For antibody-based detection, the level of the polypeptide is compared with the level of polypeptide found in a corresponding “normal” tissue.

In particular embodiments, oligonucleotide primers are based on the coding sequence region of the promoter in the CHFR gene, and are useful for amplifying DNA, for example by PCR.

The CHFR gene encodes a polypeptide that induces an early G2/M checkpoint in response to mitotic stress. Cell lines expressing wild-type CHFR exhibit low mitotic index (percentage of cells with condensed chromosomes) and delayed entry into metaphase when centrosome separation is inhibited by mitotic stress. In contrast, cancer cell lines lacking CHFR function enter metaphase without delay and demonstrate higher mitotic indices compared to the CHFR expressing cell lines. In vitro studies suggest that the RING finger domain in CHFR also facilitates ubiquitin ligase function and that it is essential for checkpoint function of CHFR. In vitro Xenopus extract experiments suggested that CHFR specifically targets PLK1 (polo-like kinase 1) for degradation when extracts are supplemented with high ubiquitin concentrations.

Disease Lack or decreased expression of CHFR is observed in a variety of cancer cell lines and tumors. Hypermethylation of the CHFR promoter is detected in a variety of cancer cell lines including esophageal, colon, lung, osteosarcoma, central nervous system, leukemic and primary tumors of the colon, lung and esophagus suggesting that decrease or loss of expression is associated with the hypermethylation of CHFR promoter.

CHFR in certain embodiments is meant to refer to the nucleic acid sequence set forth by NCBI accession No. NM_{—}018223 (SEQ ID NO: 1), and the corresponding amino acid sequence set forth NCBI accession No. NP_{—}060693 (SEQ ID NO: 2).

| SEQ ID NO: 1 | |
|---|---|
| 1 | ctcttgacag cggcggcggc gcagccggtt ccgggttcgg cgcggggcgg ggatgtgaat |
| 61 | cccgatggag cggcccgagg aaggcaagca gtcgccgccg ccgcagccct ggggacggct |
| 121 | cctgcgtctg ggcgcggagg agggcgagcc gcacgtcctc ctgaggaagc gggagtggac |
| 181 | catcgggcgg agacgaggtt gcgacctttc cttccccagc aataaactgg tctctggaga |
| 241 | tcactgtaga attgtagtgg atgaaaaatc aggtcaggtg acactggaag ataccagcac |
| 301 | cagtggaaca gtgattaaca agctgaaggt tgttaagaag cagacatgcc ctttacagac |
| 361 | tggggatgtc atctacttgg tgtacaggaa gaatgaaccg gaacacaacg tggcatacct |
| 421 | ctatgaatct ttaagtgaaa agcaaggcat gacacaagaa tcctttgaga tggtgccttg |
| 481 | ctgtgttgcc caggctggtc taaaactcct gggatcaagt gatcctccca ccttggcctc |
| 541 | ccaaagtatt gtgattacag ggtctggggg tggtggcatc tcccctaaag gaagtggtcc |
| 601 | ctctgtggca agtgatgaag tctccagctt tgcctcagct ctcccagaca gaaagactgc |
| 661 | gtccttttcg tcgttggaac cccaggatca ggaggatttg gagcccgaga agaagaaaat |
| 721 | gagaggagat ggggaccttg acctgaacgg gcagttgttg gtcgcacaac cgcgtagaaa |
| 781 | tgcccaaacc gtccacgagg acgtcagagc agcggctggg aagccagaca agatggagga |
| 841 | gacgctgaca tgcatcatct gccaggacct gctgcacgac tgcgtgagtt tgcagccctg |
| 901 | catgcacacg ttctgcgcgg cttgctactc gggctggatg gagcgctcgt ccctgtgtcc |
| 961 | tacctgccgc tgtcccgtgg agcggatctg taaaaaccac atcctcaaca acctcgtgga |
| 1021 | agcatacctc atccagcatc cagacaagag tcgcagtgaa gaagatgtgc aaagtatgga |
| 1081 | tgccaggaat aaaatcactc aagacatgct gcagcccaaa gtcaggcggt ctttttctga |
| 1141 | tgaagaaggg agttcagagg acctgctgga gctgtcagac gttgacagtg agtcctcaga |
| 1201 | cattagccag ccatacgtcg tgtgccggca gtgtcctgag tacagaaggc aggcggcgca |
| 1261 | gcctccccac tgcccagcac ccgagggcga gccaggagcc ccacaggccc tgggggatgc |
| 1321 | accccccacg tccgtcagcc tgacgacagc agtccaggat tacgtgtgcc ctctgcaagg |
| 1381 | aagccacgcc ctgtgcacct gctgcttcca gcccatgccc gaccggagag cggagcgcga |
| 1441 | gcaggacccg cgtgtcgccc ctcagcagtg tgcggtctgc ctgcagcctt tctgccacct |
| 1501 | gtactggggc tgcacccgga ccggctgcta cggctgcctg gccccgtttt gtgagctcaa |
| 1561 | cctgggtgac aagtgtctgg acggcgtgct gaacaacaac agctacgagt cagacatcct |
| 1621 | gaagaattac ctggcaacca gaggtttgac atggaaaaac atgttgaccg agagcctcgt |
| 1681 | ggctctccag cggggagtgt ttctgctgtc tgattacaga gtcacgggag acaccgttct |
| 1741 | gtgttactgc tgtggcctgc gcagcttccg tgagctgacc tatcagtatc agcagaacat |
| 1801 | tcctgcttcc gagttgccag tggccgtaac atcccgtcct gactgctact ggggccgtaa |
| 1861 | ctgccgcact caggtgaaag ctcaccacgc catgaaattc aatcatatct gtgaacagac |
| 1921 | aaggttcaaa aactaagcat ccagaggccc tgagcagctt tcagcactgg aggtgaagag |
| 1981 | agcgtgtttt taaaatacag aggcaagcac gtcaaggtgt tttcacagcc ccctgaggga |
| 2041 | agggacgcag ggtctccgac aggtgctctg gggtgactct tctgtggagc tttaccctct |
| 2101 | gagtgagacc ctccccagag ccccgggggc cgcagcccgc cctcctggtg agcgctgggc |
| 2161 | agggctcgtg gtggcatcag cagcagagac gaagcctttc tgtaacatgc ggccgtcctg |
| 2221 | ccgagagggg cagttttgct cttttgtaca ttttccgaaa ctacagttaa agcggaagtc |
| 2281 | tgttttcagg aaaagtttca agggagaagg gcaagtttat caaaaacatt gtttcaggag |
| 2341 | aagggagcat aagtttacag cctacaggac gtacacaata tcctgctgct gggaaaacca |
| 2401 | cagcatttta tctatttttt attttaatag gtttggtgct tatcttctaa taagatttaa |
| 2461 | atgtcacaaa ctgtagcaca aataatataa tttataattt acaaattgac taaaattggg |
| 2521 | tatagtatgg tatttgaaag aataagcata tgcttctgtt tattaaaaaa agaaaccttc |
| 2581 | caatgtccaa aactgctaac cctcgacgtg gccgccaagt tagtcgctcc ttgctaaccg |
| 2641 | gtgagtgacc gcggccccga gcctggggct ggacgcaggt cccaggacat gctgctccct |
| 2701 | tgtgtgagtg accgcggccc cgagcctggg gctggacgca ggtcccagga cgtgctgctc |
| 2761 | ccttgtgtga gtgaccacgg ccccaagccc agggctggag gcaggtccca ggacgcgccg |
| 2821 | ctccctcatg ctgcccgggc ccttcctcca agaccctaca gagcctgagg ggcaccttgg |
| 2881 | cttccgcctg tgctagcttt gccatgtcat ctggaataat acttgaaatt ttgattcttg |
| 2941 | gaaaaaaaag tttcttatct tttgttgaaa tcacctgtta tccttgtttg taaactgata |
| 3001 | acttttttgc ttcttctcag gaatacagtt ttcaactgtt gtcttgctct tgatagaaac |
| 3061 | tgagaagcag caatctgtat ttgtggagga aagtcctctc ttttgcatat tctaataaat |
| 3121 | gagccgcgtt tgctcctc |
| SEQ ID NO: 2 | |
| 1 | merpeegkqs pppqpwgrll rlgaeegeph vllrkrewti grrrgcdlsf psnklvsgdh |
| 61 | crivvdeksg qvtledtsts gtvinklkvv kkqtcplqtg dviylvyrkn epehnvayly |
| 121 | eslsekqgmt qesfemvpcc vaqaglkllg ssdpptlasq sivitgsggg gispkgsgps |
| 181 | vasdevssfa salpdrktas fsslepqdqe dlepekkkmr gdgdldlngq llvaqprrna |
| 241 | qtvhedvraa agkpdkmeet ltciicqdll hdcvslqpcm htfcaacysg wmersslcpt |
| 301 | crcpverick nhilnnlvea yliqhpdksr seedvqsmda rnkitqdmlq pkvrrsfsde |
| 361 | egssedllel sdvdsessdi sqpyvvcrqc peyrrqaaqp phcpapegep gapqalgdap |
| 421 | ptsvslttav qdyvcplqgs halctccfqp mpdrraereq dprvapqqca vclqpfchly |
| 481 | wgctrtgcyg clapfcelnl gdkcldgvln nnsyesdilk nylatrgltw knmlteslva |
| 541 | lqrgvfllsd yrvtgdtvlc yccglrsfre ltyqyqqnip aselpvavts rpdcywgrnc |
| 601 | rtqvkahham kfnhiceqtr fkn |

These genes are merely listed as examples and are not meant to be limiting.

Any specimen containing a detectable amount of polynucleotide or antigen can be used. Preferably the subject is human.

The present invention assesses the impact of DNA methylation in certain tumors on the sensitivity to agents directed at the microtubule, in particular the taxanes.

Using the methods of the invention, expression of CHFR can be identified in a cell and the appropriate course of treatment can be employed (e.g., microtubule-directed therapy).

Any of the methods as described herein can be used in high throughput analysis of DNA methylation. For example, U.S. Pat. No. 7,144,701, incorporated by reference in its entirety herein, describes differential methylation hybridization (DMH) for a high-throughput analysis of DNA methylation.

### II. METHODS

As described herein, the present invention features methods for identifying a subject that will respond to one or more microtubule-directed therapies. In preferred embodiments, the methods comprise detecting nucleic acid methylation of the checkpoint with forkhead and ring finger domains (CHFR) gene in one or more samples, wherein detecting nucleic acid methylation identifies a subject that will respond to one or more microtubule-directed therapies.

In preferred embodiments, the subject has been diagnosed with cancer, for example esophageal cancer, lung cancer, colon cancer, gastric cancer, and head and neck cancer. Any tumor that has CHFR methylation may be predicted to be sensitive to microtubule-directed therapies, e.g. taxane. In preferred embodiments of the invention, the tumor is an esophageal tumor.

The methods described herein feature treatment with microtubule-directed therapies. Exemplary microtubule-directed therapies include, but are not limited to, taxanes, and vinka alkaloids. Taxanes are a family of drugs that have been shown to be effective in treating various types of cancer. The principal mechanism of the taxane class of drugs is the disruption of microtubule function. Vinka Alkaloids act by binding to tubulin. Vincristine, Vinblastine are examples.

The methods of the invention can be used to predict survival in a subject with cancer, or to identify a risk of developing cancer in a subject that was treated with a microtubule-directed agent. In preferred embodiments, the method comprises detecting nucleic acid methylation of CHFR in one or more samples, and wherein detecting nucleic acid methylation identifies survival in a subject.

In any of the methods described, the subject may have previously been treated for cancer. The subject underwent previous treatment with a microtubule-directed therapy.

The methods described herein may be used to determine a course of treatment for a subject.

The invention also features methods for identifying a subject that will respond to one or more microtubule-directed therapies, predicting survival in a subject with cancer, or identifying a risk of developing cancer in a subject that was treated with a microtubule-directed therapy.

These methods comprise extracting nucleic acid from one or more cell or tissue samples, detecting nucleic acid methylation of the CHFR gene in the sample; and identifying the nucleic acid methylation state of the CHFR gene, wherein nucleic acid methylation of CHFR indicates the subject that will respond to one or more microtubule-directed therapies, or predicts survival in a subject with cancer, or identifies a risk of developing cancer in a subject that was treated with a microtubule-directed therapy.

The samples, in certain embodiments, can be from one or more of blood, blood plasma, serum, cells, a cellular extract, a cellular aspirate, tissues, a tissue sample, or a tissue biopsy.

As described herein, in certain preferred examples the genes comprise one or more CpG islands in the promoter regions. Accordingly, any gene that contains one or more CpG island in the promoter region is suitable for use in the methods of the invention; however in certain preferred examples, the one or more genes may be selected from any of the genes described in the application herein, e.g. CHFR.

Methods of Treatment

The invention as described herein can be used to treat a subject having or at risk for having cancer. Accordingly, the method comprises identifying nucleic acid methylation of a CHFR gene, and administering to the subject a therapeutically effective amount of a demethylating agent, thereby treating a subject having or at risk for having cancer.

The method can be used in combination with one or more microtubule-directed therapies as described herein.

The method can be used in combination with one or more chemotherapeutic agents. Anti-cancer drugs that may be used in the various embodiments of the invention, including pharmaceutical compositions and dosage forms and kits of the invention, include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine, mechlorethamine oxide hydrochloride rethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, improsulfan, benzodepa, carboquone, triethylenemelamine, triethylenephosphoramide, triethylenethiophosphoramide, trimethylolomelamine, chlornaphazine, novembichin, phenesterine, trofosfamide, estermustine, chlorozotocin, gemzar, nimustine, ranimustine, dacarbazine, mannomustine, mitobronitol, aclacinomycins, actinomycin F(1), azaserine, bleomycin, carubicin, carzinophilin, chromomycin, daunorubicin, daunomycin, 6-diazo-5-oxo-1-norleucine, doxorubicin, olivomycin, plicamycin, porfiromycin, puromycin, tubercidin, zorubicin, denopterin, pteropterin, 6-mercaptopurine, ancitabine, 6-azauridine, carmofur, cytarabine, dideoxyuridine, enocitabine, pulmozyme, aceglatone, aldophosphamide glycoside, bestrabucil, defofamide, demecolcine, elfornithine, elliptinium acetate, etoglucid, flutamide, hydroxyurea, lentinan, phenamet, podophyllinic acid, 2-ethylhydrazide, razoxane, spirogermanium, tamoxifen, taxotere, tenuazonic acid, triaziquone, 2,2′,2″-trichlorotriethylamine, urethan, vinblastine, vincristine, vindesine and related agents. 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cisporphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; taxel; taxel analogues; taxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. Preferred additional anti-cancer drugs are 5-fluorouracil and leucovorin. Additional cancer therapeutics include monoclonal antibodies such as rituximab, trastuzumab and cetuximab.

Demethylating Agents

In certain embodiments, the invention features methods of identifying an agent that de-methylates methylated nucleic acids comprising identifying one or more cell or tissue samples with methylated nucleic acid, extracting the methylated nucleic acid, contacting the nucleic acid with one or more nucleic acid de-methylating candidate agents and a control agent, and identifying the nucleic acid methylation state, wherein nucleic acid de-methylation of genes in the sample by the candidate agent compared to the control indicates a demethylating agent, thereby identifying an agent that de-methylates methylated nucleic acid.

Demethylating agents include, but are not limited to, 5-aza-2′-deoxycytidine, 5-aza-cytidine, Zebularine, procaine, and L-ethionine.

Another way to restore epigenetically silenced gene expression is to introduce a non-methylated polynucleotide into a cell, so that it will be expressed in the cell. Various gene therapy vectors and vehicles are known in the art and any can be used as is suitable for a particular situation. Certain vectors are suitable for short term expression and certain vectors are suitable for prolonged expression. Certain vectors are trophic for certain organs and these can be used as is appropriate in the particular situation. Vectors may be viral or non-viral. The polynucleotide can, but need not, be contained in a vector, for example, a viral vector, and can be formulated, for example, in a matrix such as a liposome, microbubbles. The polynucleotide can be introduced into a cell by administering the polynucleotide to the subject such that it contacts the cell and is taken up by the cell and the encoded polypeptide expressed. Preferably the specific polynucleotide will be one which the patient has been tested for and been found to carry a silenced version.

### III. Samples

Samples for use in the methods of the invention include cells or tissues obtained from any solid tumor, samples taken from blood, blood plasma, serum, cells, a cellular extract, a cellular aspirate, tissues, a tissue sample, or a tissue biopsy. Tumor DNA can be found in various body fluids and these fluids can potentially serve as diagnostic material.

In preferred embodiments, the tumor sample is selected from tumors that show CHFR methylation. Exemplary tumors include esophageal, lung, colon, gastric, and head and neck. In preferred embodiments, the tumor is an esophageal tumor.

Any nucleic acid specimen, in purified or nonpurified form, can be utilized as the starting nucleic acid or acids, provided it contains, or is suspected of containing, the specific nucleic acid sequence containing the target locus (e.g., CpG). Thus, the process may employ, for example, DNA or RNA, including messenger RNA, wherein DNA or RNA may be single stranded or double stranded. In the event that RNA is to be used as a template, enzymes, and/or conditions optimal for reverse transcribing the template to DNA would be utilized. In addition, a DNA-RNA hybrid which contains one strand of each may be utilized. A mixture of nucleic acids may also be employed, or the nucleic acids produced in a previous amplification reaction herein, using the same or different primers may be so utilized. The specific nucleic acid sequence to be amplified, i.e., the target locus, may be a fraction of a larger molecule or can be present initially as a discrete molecule, so that the specific sequence constitutes the entire nucleic acid. It is not necessary that the sequence to be amplified be present initially in a pure form; it may be a minor fraction of a complex mixture, such as contained in whole human DNA.

The nucleic acid-containing sample or specimen used for detection of methylated CpG may be extracted by a variety of techniques such as that described by Maniatis, et al. (Molecular Cloning: A Laboratory Manual, Cold Spring Harbor, N.Y., pp 280, 281, 1982).

If the extracted sample is impure (e.g., plasma, serum, stool, ejaculate, sputum, saliva, ductal cells, nipple aspiration fluid, ductal lavage fluid, cerebrospinal fluid or blood or a sample embedded in paraffin), it may be treated before amplification with an amount of a reagent effective to open the cells, fluids, tissues, or animal cell membranes of the sample, and to expose and/or separate the strand(s) of the nucleic acid(s). This lysing and nucleic acid denaturing step to expose and separate the strands will allow amplification to occur much more readily

Preferably, the method of amplifying is by PCR, as described herein and as is commonly used by those of ordinary skill in the art. However, alternative methods of amplification have been described and can also be employed. PCR techniques and many variations of PCR are known. Basic FUR techniques are described by Saiki et al. (1988 Science 239:487-491) and by U.S. Pat. Nos. 4,683,195, 4,683,202 and 4,800,159, each of which is incorporated herein by reference.

The conditions generally required for PCR include temperature, salt, cation, pH and related conditions needed for efficient copying of the master-cut fragment. PCR conditions include repeated cycles of heat denaturation (i.e. heating to at least about 95 C.) and incubation at a temperature permitting primer: adaptor hybridization and copying of the master-cut DNA fragment by the amplification enzyme. Heat stable amplification enzymes like the pwo, Thermus aquaticus or Thermococcus litoralis DNA polymerases which eliminate the need to add enzyme after each denaturation cycle, are commercially available. The salt, cation, pH and related factors needed for enzymatic amplification activity are available from commercial manufacturers of amplification enzymes.

As provided herein an amplification enzyme is any enzyme which can be used for in vitro nucleic acid amplification, e.g. by the above-described procedures. Such amplification enzymes include pwo, Escherichia coli DNA polymerase I, Klenow fragment of E. coli polymerase I, T4 DNA polymerase, T7 DNA polymerase, Thermus aquaticus (Taq) DNA polymerase, Thermococcus litoralis DNA polymerase, SP6 RNA polymerase, T7 RNA polymerase, T3 RNA polymerase, T4 polynucleotide kinase, Avian Myeloblastosis Virus reverse transcriptase, Moloney Murine Leukemia Virus reverse transcriptase, T4 DNA ligase, E. coli DNA ligase or Q.beta. replicase. Preferred amplification enzymes are the pwo and Taq polymerases. The pwo enzyme is especially preferred because of its fidelity in replicating DNA.

Once amplified, the nucleic acid can be attached to a solid support, such as a membrane, and can be hybridized with any probe of interest, to detect any nucleic acid sequence. Several membranes are known to one of skill in the art for the adhesion of nucleic acid sequences. Specific non-limiting examples of these membranes include nitrocellulose (NITROPURE) or other membranes used in for detection of gene expression such as polyvinylchloride, diazotized paper and other commercially available membranes such as GENESCREEN, ZETAPROBE . . . (Biorad), and NYTRAN. Methods for attaching nucleic acids to these membranes are well known to one of skill in the art. Alternatively, screening can be done in a liquid phase.

In nucleic acid hybridization reactions, the conditions used to achieve a particular level of stringency will vary, depending on the nature of the nucleic acids being hybridized. For example, the length, degree of complementarity, nucleotide sequence composition (e.g., GC v. AT content), and nucleic acid type (e.g., RNA v. DNA) of the hybridizing regions of the nucleic acids can be considered in selecting hybridization conditions. An additional consideration is whether one of the nucleic acids is immobilized, for example, on a filter.

An example of progressively higher stringency conditions is as follows: 2.times.SSC/0.1% SDS at about room temperature (hybridization conditions); 0.2.times.SSC/0.1% SDS at about room temperature (low stringency conditions); 0.2.times.SSC/0.1% SDS at about 42° C. (moderate stringency conditions); and 0.1.times.SSC at about 68° C. (high stringency conditions). Washing can be carried out using only one of these conditions, e.g., high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically. In general, conditions of high stringency are used for the hybridization of the probe of interest.

The probe of interest can be detectably labeled, for example, with a radioisotope, a fluorescent compound, a bioluminescent compound, a chemiluminescent compound, a metal chelator, or an enzyme. Those of ordinary skill in the art will know of other suitable labels for binding to the probe, or will be able to ascertain such, using routine experimentation.

### IV. Kits

The methods of the invention are ideally suited for the preparation of kits.

The invention features kits for identifying the nucleic acid methylation state of CHFR comprising gene specific primers for use in polymerase chain reaction (PCR), and instructions for use.

The invention also features kits for detecting the sensitivity of a subject to microtubule based therapy by detecting nucleic acid methylation of CHFR, the kit comprising gene specific primers for use in polymerase chain reaction (PCR), and instructions for use.

As described above, the PCR, in particularly preferred examples, is methylation specific PCR (MSP).

In certain embodiments, any gene comprising one or more CpG islands in the promoter region can be detected using the kits of the invention. In certain preferred examples, the gene is CHFR.

Carrier means are suited for containing one or more container means such as vials, tubes, and the like, each of the container means comprising one of the separate elements to be used in the method. In view of the description provided herein of invention methods, those of skill in the art can readily determine the apportionment of the necessary reagents among the container means. For example, one of the container means can comprise a container containing gene specific primers for use in polymerase chain reaction methods of the invention. In addition, one or more container means can also be included which comprise a methylation sensitive restriction endonuclease.

The following examples are offered by way of illustration, not by way of limitation. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

### EXAMPLES

The present experiments examine the DNA methylation of the CHFR gene in esophageal cancer patients. Previous in vitro studies suggested differential sensitivity to microtubule-directed agents. The results show that CHFR methylation is a predictive biomarker for sensitivity to taxane-based chemoradiotherapy and is a determinant of survival in treatment of esophageal cancer patients with microtubule-directed agents.

### Example 1

### Patient Characteristics

Endoscopic biopsies from patients with newly diagnosed esophageal cancer were obtained to determine the relationship between CHFR methylation and response and survival in patients treated with taxane and non-taxane containing regimens (Table 1, below).

TABLE 1

| Chemotherapy | Number of Patients |
|---|---|
| Taxane treated patients | |
| Docetaxel/5-Fluorouracil | 9 |
| Docetaxel/5-Fluorouracil/Cisplatin | 9 |
| Paclitaxel/5-Flurouracil/Cisplatin | 14 |
| Paclitaxel/Cisplatin | 11 |
| Paclitaxel/Cisplatin/Gefitinib | 5 |
| Paclitaxel/Carboplatin | 1 |
| Total | 49 |
| Non-taxane treated patients | |
| 5-Fluorouracil | 2 |
| 5-Fluorouracil/Cisplatin | 16 |
| 5-Fluorouracil/Oxaliplatin | 3 |
| Cisplatin/Irinotecan | 11 |
| Total | 32 |

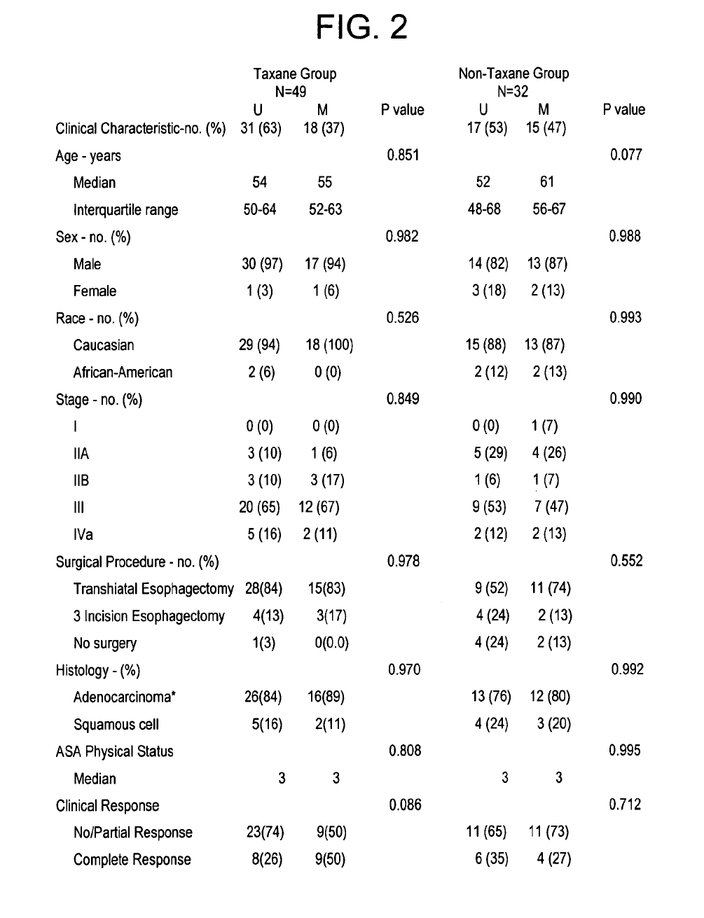

Clinical and demographic variables were similar in patients treated with a taxane or non-taxane regimens, and for methylated and unmethylated tumors (FIG. 2). FIG. 2 is a Table showing the distribution of CHFR methylation according to taxane treatment.

Using the American Society of Anesthesia Physical Status Classification, patients in both taxane and non-taxane exposed groups were found equally fit for surgery.

### Example 2

### Analysis of CHFR Methylation by Gel-Based MSP and Development of a Quantitative MSP Assay

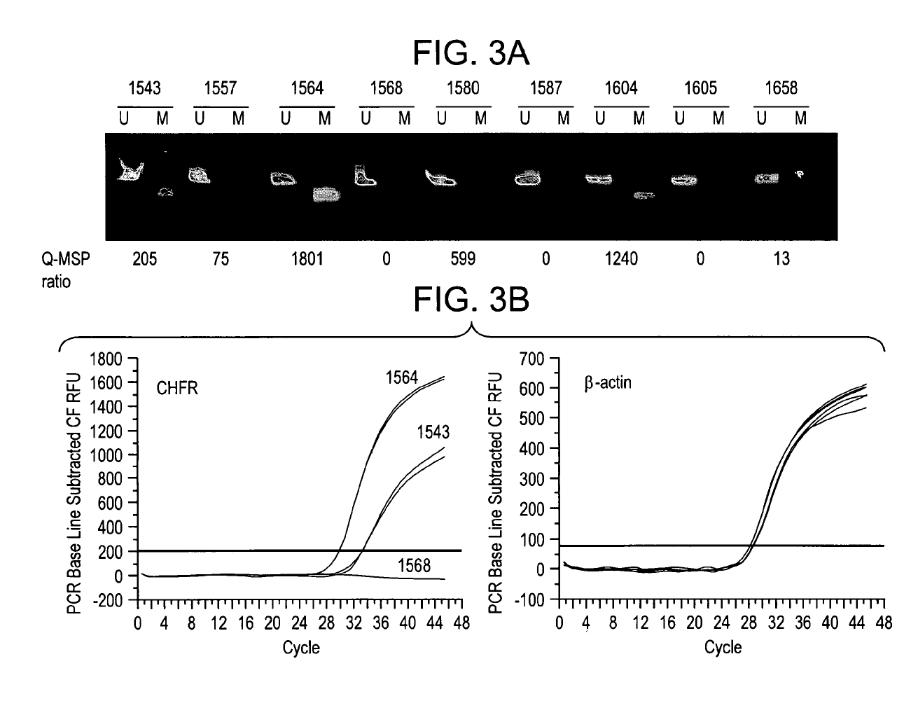

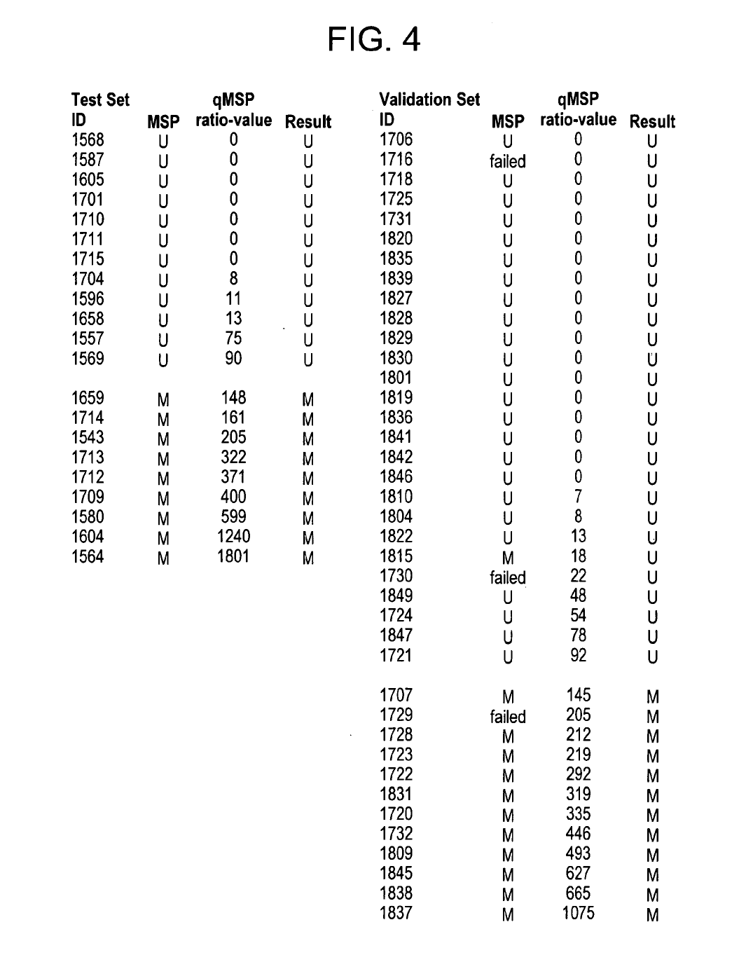

An initial test set of twenty-one patients exposed to combination therapy with taxanes was examined using gel-based MSP for CHFR. CHFR methylation was detected in 10 of 21 patients (48%, examples in FIG. 3). Within this test set, a trend towards the likelihood of achieving a pCR was found for patients with CHFR methylation (Odds ratio 4.00, 954% Cl 0.64-25.02, p=0.14). To facilitate the use of CHFR methylation in clinical laboratories and extend these findings to reduce this large confidence interval, a real-time CHFR MSP analysis was developed that eliminates the need for electrophoresis gels 21. A cut-off value of 100 for the determination of methylation was derived from a comparison between gel-based MSP and qMSP results in the original twenty-one patients (FIG. 3 and Table in FIG. 4). FIG. 4 shows methylation of CHFR determined by gel based and real time qMSP.

Next, an additional twenty-eight patients exposed to taxane therapy and non taxane treated patients were analyzed using gel-based MSP and qMSP. There was one case of discordance and three cases where gel-based amplification failed. In the cases of failed amplification, low amounts or poor quality DNA from paraffin-tissue led to insufficient visualization of PCR products on gel analysis. In contrast, the single discordant result was a negative result on qMSP (low level CHFR methylation) in a sample with abundant DNA resulting in detection in gel-based analysis interpreted as positive. The frequency of CHFR methylation was 41% for the entire cohort of 81 patients, with no significant difference in CHFR methylation incidence according to treatment [Taxane exposed 37% (18/49) vs. non-exposed 47% (15/32): p=0.49]. CHFR methylation was not associated with patient gender, age, performance status or tumor stage or histology (FIG. 2).

### Example 3

### CHFR Methylation Predicts Survival after Combination Treatment with Taxanes

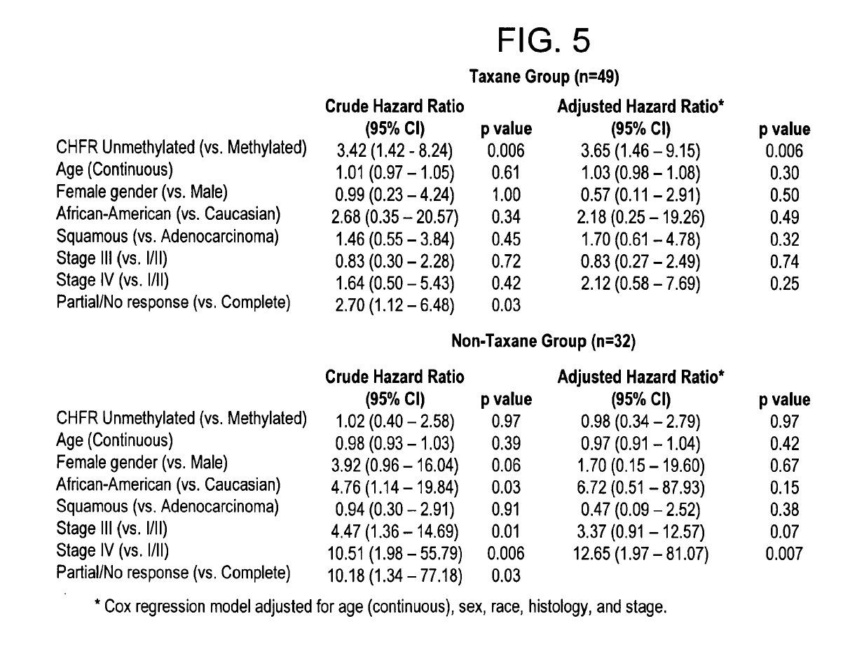

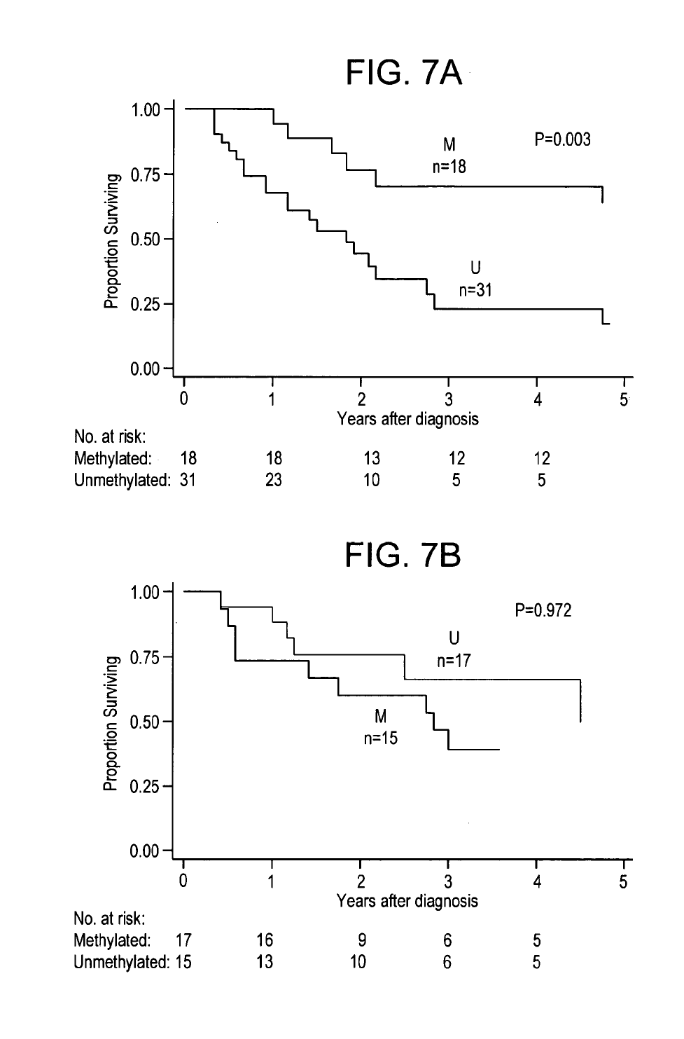

The results of real-time methylation analysis of CHFR were compared to overall survival in the 49 patients treated with taxanes. In this population, CHFR methylation continued to predict a trend towards achieving a pCR (Odds ratio 2.88, CI 0.84-9.78, p=0.09). However, the strongest correlation was between CHFR methylation and overall survival. In univariate analysis, patients with tumors having unmethylated vs. methylated CHFR had a crude hazard ratio for death of 3.42 (95% Cl 1.42-8.24, p=0.006, Table in FIG. 5). Kaplan-Meier curves for overall survival demonstrate a statistically significant difference (p=0.003) according to CHFR methylation status (FIG. 7A), with a median survival for unmethylated patients of 22 months (IQR 8-34) and not yet reached for methylated patients. The 2 and 5-year survival rates were longer in patients with CHFR methylation (77% and 64% respectively), compared to patients without CHFR methylation (44% and 17% respectively). Disease specific survival was associated with the same survival advantage for CHFR methylation (data not shown), since deaths were associated with disease recurrence and progression.

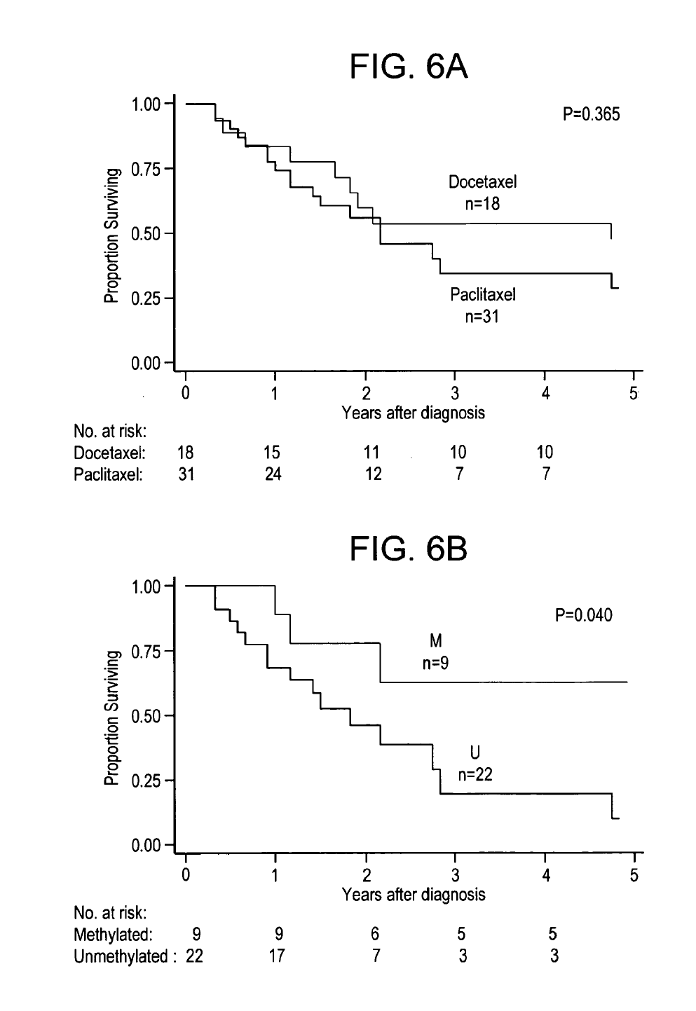

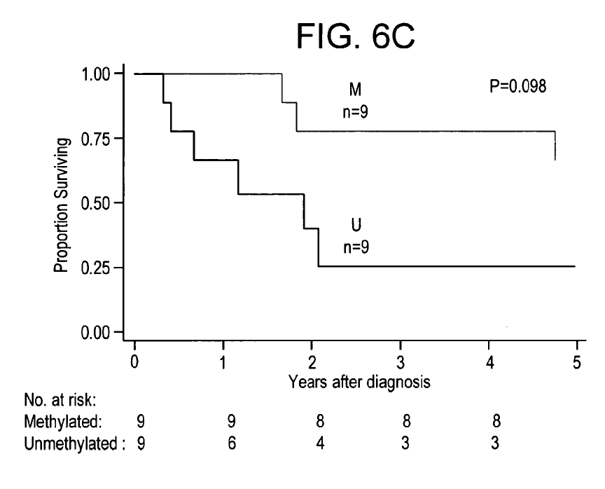

To account for potential confounding factors associated with survival, age, race, sex and stage were adjusted for in the multivariate Cox regression model. CHFR methylation remained predictive of overall survival, with an adjusted hazard ratio for death of 3.65 (95% Cl 1.46-9.15, p=0.006). There was no difference in survival between patients treated with docetaxel vs. paclitaxel (FIG. 5 and FIG. 6A). However, the survival advantage for CHFR methylation was seen for both paclitaxel (FIG. 6B) and docetaxel (FIG. 6C), suggesting that CHFR methylation is predictive for survival to the taxane class rather than to individual agents.

To determine whether CHFR methylation was truly predictive of taxane-based responses and survival or was a prognostic marker irrespective of therapy, CHFR methylation was examined in a control group of 32 patients who did not receive taxanes. For these patients, there was no difference in survival according to CHFR methylation status (FIG. 7B and FIG. 5, crude hazard ratio of death for unmethylated CHFR 1.02 (95% Cl 0.40-2.58; p=0.97), adjusted hazard ratio 0.98 (95% Cl 0.34-2.79, p=0.97). These data verify in vitro evidence that CHFR methylation is a specific predictor for taxane sensitivity and not chemosensitivity in general.

### Example 4

### CHFR Methylation Predicts Survival Following Complete Pathologic Response

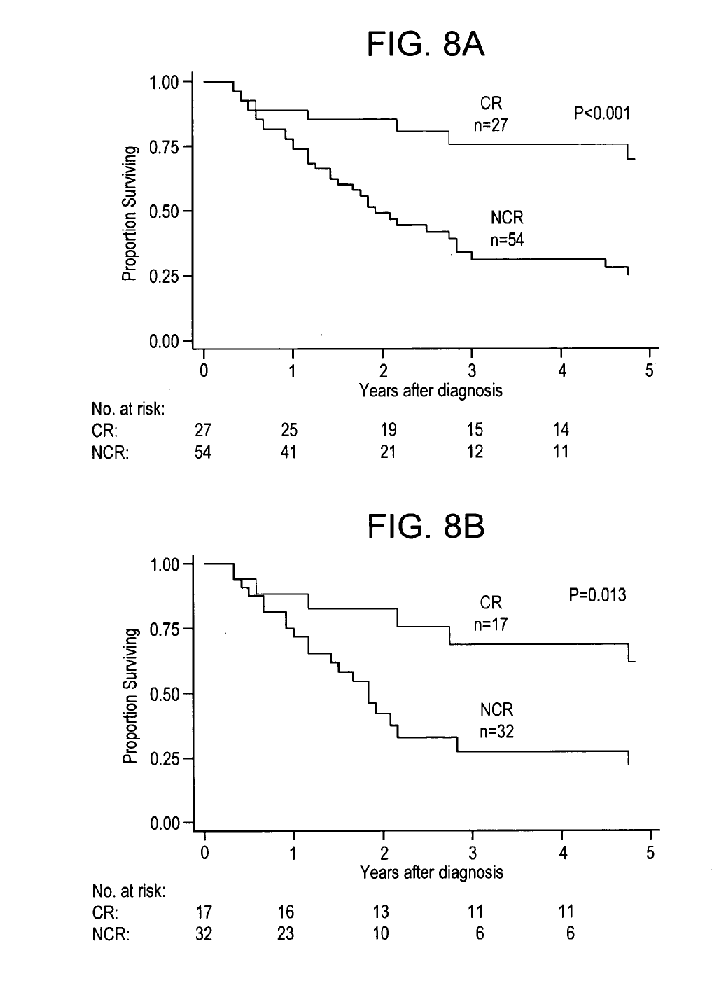

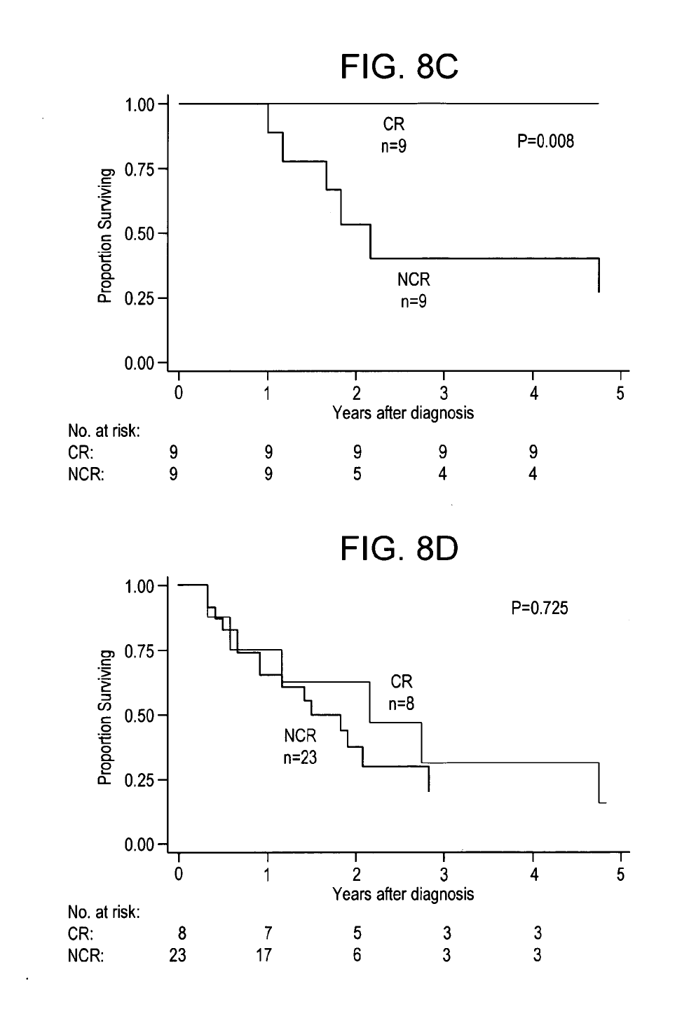

Complete pathologic response has been the best predictor of long-term survival after neoadjuvant therapy for esophageal cancer 22, 23. A pCR was achieved in 27 of the 81 patients (33%) treated with preoperative chemoradiotherapy. The median survival for patients achieving a pCR was not reached, significantly better than for patients with less than pathologic CR after neoadjuvant therapy (23 months, 95% CI 12-57, p=0.024). In the entire cohort, 5 year survival for patients achieving a pCR was 70% versus 25% for those patients not achieving a pCR (FIG. 8A). With taxane-based therapy, 5 year survival for the 17 patients (35%) achieving a pCR was 62% (FIG. 8B), but was only 22% in 32 patients not achieving a pCR. The odds ratio of achieving a pCR for taxane treated patients with CHFR methylation was 2.88 (95% Cl 0.84-9.79; p=0.09), but was 0.67 (95% Cl 0.14-3.13; p=0.61) for non taxane treated patients. Importantly, all patients with CHFR methylation in tumor achieving a pCR remained disease-free 5 years after surgery (median survival not reached), (FIG. 8C), while those not achieving a pCR had a median survival of 26 months. While patients with unmethylated tumors achieving a pCR survived longer than those without pCR (26 vs. 18 months) (FIG. 3D), the median survival was no better than patients with a methylated CHFR not achieving a pCR.

Preoperative chemoradiation therapy followed by surgery is central in the definitive treatment for advanced localized esophageal cancer. The most frequently used regimen of cisplatin/5-FU has observed pCR rates of 30%. Recent trials incorporating newer agents into the treatment schema, such as paclitaxel, have demonstrated similar or lower pCR rates. 24, 25 To achieve improvements in response rates and outcome, individualized therapy based on the molecular makeup of the tumor may be beneficial. Therefore, in this study, we tested the hypothesis that promoter methylation of the mitotic checkpoint gene CHFR can serve as a predictive marker to identify patients more likely to respond to neoadjuvant taxane containing regimens.

The results presented herein suggest that CHFR methylation in pre-treatment biopsies of esophageal cancer predicts clinical response and overall survival in patients treated with taxane-based chemotherapy, supporting in vitro observations that CHFR inactivation specifically predicts sensitivity to taxanes and not to chemotherapy in general. The observed 50% pCR rate in patients with CHFR methylation also suggests that the inability of previous clinical trials to show improved tumor response with taxane based preoperative chemoradiation in unselected patients may be due to accrual of many patients without CHFR methylation who are taxane resistant.

Several lines of external validation exist for the finding presented herein: Patient characteristics and comparable overall survival rates of the cohort compare well with historical controls 1, thus validating its composition. The finding that CHFR methylation in the absence of treatment with taxanes is not a prognostic marker has been described independently 26, while in vitro studies suggest specific sensitivity to taxanes associated with CHFR inactivation 14, 16. The present inventors have previously demonstrated for MGMT that detection of DNA methylation correlates better with response to alkyating agents 3 than protein expression assessed by immunohistochemistry 27 or enzyme activity 28. This is in part due to the challenges associated with quantitative assessment using immunohistochemistry and the presence of normal cells in a tumor which has no influence on the detection of methylation in malignant cells but which affects the overall mRNA or protein expression detection 3.

The observed discordance in survival in taxane exposed patients with CHFR methylated and unmethylated tumors who did or did not achieve a pCR is of particular interest: CHFR methylated patients who achieved a pCR had a high chance for cure from esophageal cancer, while there was no considerable survival difference between unmethylated patients who did or did not achieve a pCR. It is likely that the most important contributor to long term survival is eradication of distant micrometastasis. It is therefore possible that in patients with CHFR unmethylated tumors, a pCR reflects primarily radiosensitivity rather than chemosensitivity. This hypothesis is supported by the observation that all cancer recurrences in these patients occurred outside the radiation port. For patients with CHFR methylated tumors, however, a highly effective strategy is the inclusion of a taxane in the treatment regimen. Moreover, even for those patients with CHFR methylated tumors not achieving pCR, both median survival and 5-year survival were better than expected, suggesting that in some patients, despite the failure to achieve a pCR, chemotherapy might have eradicated distant metastasis. These patients appear to have overall survival similar to patients with CHFR unmethylated tumors achieving a pCR, resulting in CHFR methylation being a strong predictor of survival than pCR in these patients.

### Methods

The invention was performed using, but not limited to, the methods as described herein.

Study Population and Tumor Specimens

Endoscopic tumor biopsy specimens were studied from 81 patients with advanced localized esophageal cancer scheduled to undergo neoadjuvant chemoradiotherapy and surgery at the Johns Hopkins Hospital (JHH) between January 1995 and January 2007. Forty-nine patients were treated with taxane based combination chemoradiotherapy. Pretreatment esophageal tumor tissue from endoscsopic biopsy was initially available from JHH for twenty-one patients treated with taxanes. After an analysis of these initial 21 patients, IRB permission was obtained to request further biopsy specimens from referring endoscopy centers for patients subsequently treated at Johns Hopkins Hospital. This allowed analysis of pretreatment endoscopic tumor biopsies from an additional 28 patients treated with taxanes and 32 patients treated with non-taxane regimens. Seven patients did not undergo resection due to medical complications or progressive/metastatic disease prior to surgery. Of the forty-nine taxane-treated patients, thirty-one received paclitaxel containing combinations, while eighteen received a docetaxel-based combination shown in the Table below. Table 1 shows chemotherapy regimens for esophageal patients (n=81).

TABLE 1

| Chemotherapy | Number of Patients |
|---|---|
| Taxane treated patients | |
| Docetaxel/5-Fluorouracil | 9 |
| Docetaxel/5-Fluorouracil/Cisplatin | 9 |
| Paclitaxel/5-Flurouracil/Cisplatin | 14 |
| Paclitaxel/Cisplatin | 11 |
| Paclitaxel/Cisplatin/Gefitinib | 5 |
| Paclitaxel/Carboplatin | 1 |
| Total | 49 |
| Non-taxane treated patients | |
| 5-Fluorouracil | 2 |
| 5-Fluorouracil/Cisplatin | 16 |
| 5-Fluorouracil/Oxaliplatin | 3 |
| Cisplatin/Irinotecan | 11 |
| Total | 32 |

Response to preoperative chemoradiotherapy was based on pathology of the resected esophagus and nodal tissue specimen and classified as either pathologic complete response (pCR) or non-pCR, where pCR is the complete absence of intact tumor cells in surgical pathology specimens. We verified histological results and deaths during follow-up by re-examining original hospital records. This study was approved by the Johns Hopkins Institutional Review Board.

Preparation of Tumor

Pre-treatment tumor specimens were obtained either as fresh tissue at the time of endoscopy without knowledge of the proposed neoadjuvant chemotherapy regimen, or from pathology archives. Specimens were labeled only with study specific identifiers; laboratory investigators had no knowledge of treatment or clinical outcome. DNA was extracted from three sequential 10 μm sections of unstained formalin-fixed or fresh frozen endoscopic tumor biopsies. For each sample, adjacent sections were stained (H&E) for histological confirmation of the presence of malignancy. DNA was extracted as previously described 20, measured spectrophotometrically and 1 μg bisulfite modified using the EZ DNA Methylation Kit (Zymo Research, Orange, Calif., USA).

Gel-Based Methylation-Specific PCR (MSP)

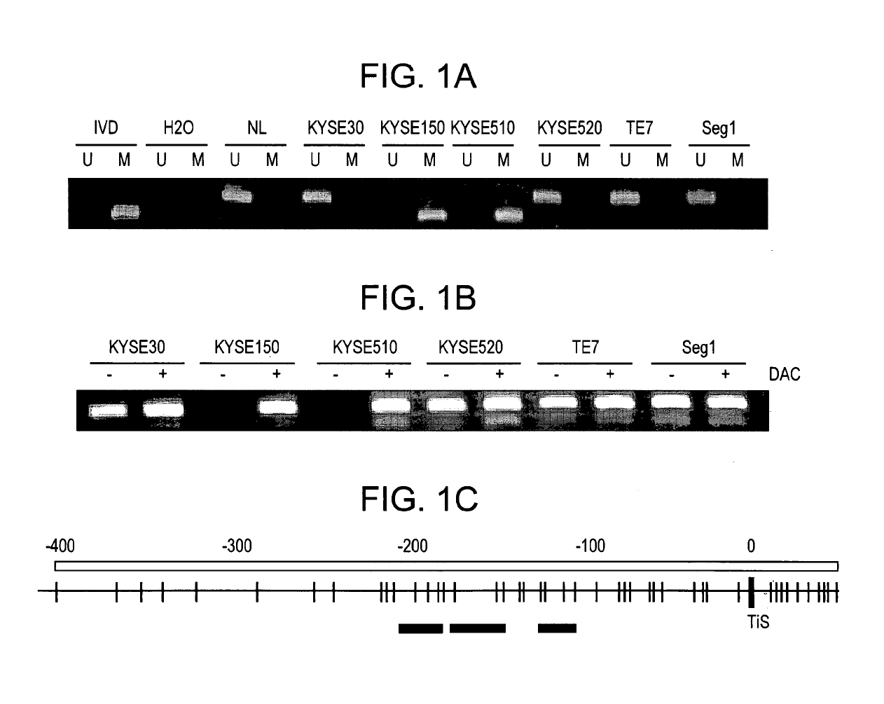

A previously described gel-based MSP assay for detection of CHFR methylation in colorectal cancer was used 20. CHFR methylation correlates with complete silencing of gene transcription in esophageal cancer cell lines (4 squamous cell and 2 adenocarcinoma cell lines) cell lines (FIG. 1A), which was reversed by treatment with the DNA methyltransferase inhibitor 5-aza-2′ deoxycytidine (FIG. 1B).

Direct, Real-Time MSP (qMSP)

Methylation of CHFR was determined by detecting analyte (CHFR and ACTS) with real-time MSP using molecular beacons. These consisted of parallel amplification/quantification using specific primer and detector pairs for each analyte using the iCycler (BioRad Laboratories, Hercules, Calif.). The CHFR promoter sequence (FIG. 1C) detects fully methylated (mCHFR), while ACTB for normalization uses methylation independent primers. The amplicon is 100 by for mCHFR and 103 by for ACTB. Duplicate PCR amplifications were analyzed using iQuant 3.0 software (BioRad), exported as Ct values, and used to calculate copy numbers based on a linear regression of values plotted on a standard curve of 20-2×10⁶ gene copy equivalents of plasmid DNA containing bisulfite modified CHFR. CHFR copy numbers were divided by ACTB copy numbers for each sample to normalize for DNA quality and quantity, multiplied by 100 for convenience to determine the ratio-value. Cut-offs were applied to this ratio-value to determine methylation status.

The CHFR target sequence is on chromosome 12 between positions 131974455 and 131974355, while the ACTB target sequence is on chromosome 7 between positions 5538428 and 5538325, (version 36.1 of the NCBI human genome).

Sequences for forward and reverse primers are:

Forward Primer CHFR:
(SEQ ID NO: 3)
5′-GTTATTTTCGTGATTCGTAGGCGAC-3′
Reverse Primer CHFR:
(SEQ ID NO: 4)
5′-CGAAACCGAAAATAACCCGCG-3′
Forward Primer ACTB:
(SEQ ID NO: 5)
5′-TAGGGAGTATATAGGTTGGGGAAGTT-3′
Reverse Primer ACTB:
(SEQ ID NO: 6)
5′-AACACACAATAACAAACACAAATTCAC-3′
Beacon CHFR:
(SEQ ID NO: 7)
5′-CGACATGCGAAGTCGTTTGGTTAGGATTAAAGATGGICGAGC
GGCATGTCG-3′
Beacon ACTB:
(SEQ ID NO: 8)
5′-CGACTGCGTGIGGGGTGGTGATGGAGGAGGTTTAGGCAGTC
G-3′

Statistical Analysis

Based upon findings from the initial 21 patients treated with taxanes and 40% methylation prevalence, the study size necessary to detect a hazard ratio of death of 3.00 for unmethylated CHFR was estimated. For >80% power with alpha <0.05, two tailed, the study population needed to be approximately 40 patients. The primary endpoint was time to death or censor measured from the origin date of pretreatment endoscopic biopsy. Subjects alive with no evidence of disease at the end of the study were censored for death. All deaths were cancer related and no subject was lost to follow-up. Associations between clinical response and CHFR methylation were assessed using logistic regression. Kaplan-Meier method was used to estimate all cause survival. Statistical comparisons and survival between groups were analyzed using the log-rank test. Associations between overall survival, prognostic factors, and exposure status were assessed using univariate and multivariate Cox proportional-hazard regression. The multivariate model for survival was adjusted using age, race, sex, and stage. Results of all models are reported as relative risks with 95% confidence intervals. All statistical calculations were performed using Stata Statistical Software, College Station, Tex. Associations were considered significant with p<0.05 (two-sided).

The present invention has been described in detail, including the preferred embodiments thereof. However, it will be appreciated that those skilled in the art, upon consideration of the present disclosure, may make modifications and/or improvements of this invention and still be within the scope and spirit of this invention as set forth in the following claims.

All publications and patent documents cited in this application are incorporated by reference in their entirety for all purposes to the same extent as if each individual publication or patent document were so individually denoted. By their citation of various references in this document, Applicants do not admit any particular reference is “prior art” to their invention.

### REFERENCES

1. Kleinberg L, Forastiere AA. Chemoradiation in the management of esophageal cancer. J Clin Oncol 2007; 25(26):4110-7.
2. Adelstein D J, Rice T W, Rybicki L A, et al. Does paclitaxel improve the chemoradiotherapy of locoregionally advanced esophageal cancer? A nonrandomized comparison with fluorouracil-based therapy. J Clin Oncol 2000; 18(10):2032-9.
3. Esteller M, Garcia-Foncillas J, Andion E, et al. Inactivation of the DNA-Repair Gene MGMT and the Clinical Response of Gliomas to Alkylating Agents. NEnglJ Med 2000; 343:1350-4.
4. Hegi M E, Diserens A C, Gorlia T, et al. MGMT gene silencing and benefit from temozolomide in glioblastoma. N Engl J Med 2005; 352(10):997-1003.
5. Scohick D M, Halazonetis T D. Chfr defines a mitotic stress checkpoint that delays entry into metaphase. Nature 2000; 406(6794):430-5.
6. Chaturvedi P, Sudakin V, Bobiak M L, et al. Chfr regulates a mitotic stress pathway through its RING-finger domain with ubiquitin ligase activity, Cancer Res 2002; 62(6):1797-801.
7. Yu X, Minter-Dykhouse K, Malureanu L, et al. Chfr is required for tumor suppression and Aurora A regulation. Nat Genet. 2005; 37(4):401-6.
8. Kang D, Chen J, Wong J, Fang G. The checkpoint protein Chfr is a ligase that ubiquitinates Plkl and inhibits Cdc2 at the G2 to M transition. J Cell Biol 2002; 156(2):249-59.
9. Summers M K, Bothos J, Halazonetis T D. The CHFR mitotic checkpoint protein delays cell cycle progression by excluding Cyclin B1 from the nucleus, Oncogene 2005; 24(16):2589-98.
10. Mizuno K, Osada H, Konishi H, et al. Aberrant hypermethylation of the CHFR prophase checkpoint gene in human lung cancers. Oncogene 2002; 21(15):2328-33.
11. Corn P G, Summers M K, Fogt F, et al. Frequent hypermethylation of the 5′ CpG island of the mitotic stress checkpoint gene Chfr in colorectal and non-small cell lung cancer. Carcinogenesis 2003; 24(1):47-51.
12. Toyota M, Sasaki Y, Satoh A, et al. Epigenetic inactivation of CHFR in human tumors. Proc Natl Acad Sci USA 2003; 100(13):7818-23.
13. Mariatos G, Bathos J, Zacharatos P, et al. Inactivating mutations targeting the chfr mitotic checkpoint gene in human lung cancer. Cancer Res 2003; 63(21):7185-9.
14. Satoh A, Toyota M, Itoh F, et al. Epigenetic inactivation of CHFR and sensitivity to microtubule inhibitors in gastric cancer. Cancer Res 2003; 63(24):8606-13.
15. Koga Y, Kitajima Y, Miyoshi A, Sato K, Sato S, Miyazaki K. The significance of aberrant CHFR methylation for clinical response to microtubule inhibitors in gastric cancer, J Gastroenterol 2006; 41(2):133-9.
16. Ogi K, Toyota M, Mita H, et al. Small interfering RNA-induced CHFR silencing sensitizes oral squamous cell cancer cells to microtubule inhibitors. Cancer Biol Ther 2005; 4(7):773-80.
17. Yanokura M, Banno K, Kawaguchi M, et al. Relationship of aberrant DNA hypermethylation of CHFR with sensitivity to taxanes in endometrial cancer. Oncol Rep 2007; 17(1):41-8.
18. Banno K, Yanokura M, Kawaguchi M, et al. Epigenetic inactivation of the CHFR gene in cervical cancer contributes to sensitivity to taxanes. Int J Oncol 2007; 31(4):713-20.
19. Yoshida K, Hamai Y, Suzuki T, Sanada Y, Oue N, Yasui W. DNA methylation of CHFR is not a predictor of the response to docetaxel and paclitaxel in advanced and recurrent gastric cancer. Anticancer Res 2006; 26(1A):49-54.
20. Brandes J C, van Engeland M, Wouters K A, Weijenberg M P, Herman J G. CHFR promoter hypermethylation in colon cancer correlates with the microsatellite instability phenotype. Carcinogenesis 2005; 26(6):1152-6.
21. Kagan J, Srivastava S; Barker P E, Belinsky S A, Cairns P. Towards Clinical Application of Methylated DNA Sequences as Cancer Biomarkers: A Joint NCl's EDRN and NIST Workshop on Standards, Methods, Assays, Reagents and Tools. Cancer Res 2007; 67(10):4545-9.
22. Kleinberg L, Gibson M K, Forastiere A A. Chemoradiotherapy for localized esophageal cancer: regimen selection and molecular mechanisms of radiosensitization. Nat Clin Pract Oncol 2007; 4(5):282-94.
23. Hammoud Z T, Kesler K A, Ferguson M K, et al. Survival outcomes of resected patients who demonstrate a pathologic complete response after neoadjuvant chemoradiation therapy for locally advanced esophageal cancer. Dis Esophagus 2006; 19(2):69-72.
24. Urba S G, Orringer M B, lanettonni M, Hayman J A, Satoru H. Concurrent cisplatin, paclitaxel, and radiotherapy as preoperative treatment for patients with locoregional esophageal carcinoma. Cancer 2003; 98(10):2177-83.
25. Kleinberg L, Powell M E, Forastiere A, Keller S, Anne P, Benson A B. E1201: An Eastern Cooperative Oncology Group randomized phase II trial of neoadjuvant preoperative paclitaxelicisplatin/RT or irinotecan/cisplatin/RT in endoscopy with ultrasound staged adenocarcinoma of the esophagus. J Clin Oncol 2007; 25(18s):205s.
26. Hamilton J P, Sato F, Greenwald B D, et al. Promoter Methylation and Response to Chemotherapy and Radiation in Esophageal Cancer. Clin Gastroenterol Hepatol 2006.
27. Jaeckle K A, Eyre H J, Townsend J J, et al. Correlation of tumor methylguanine-DNA methyltransferase levels with survival of malignant astrocytoma patients treated with bis-chloroethylnitrosourea: a Southwest Oncology Group study. JClinOncol 1998; 16:3310-5.
28. Silber J R, Bobola M S, Ghatan S, Blank A, Kolstoe D D, Berger M S. methylguanine-DNA methyltransferase activity in adult gliomas: relation to patient and tumor characteristics. Cancer Res 1998; 58:1068-73.

1. A method for identifying a subject that will respond to one or more microtubule-directed therapies comprising: detecting nucleic acid methylation of the checkpoint with forkhead and ring finger domains (CHFR) gene in one or more samples, wherein detecting nucleic acid methylation identifies a subject that will respond to one or more microtubule-directed therapies.

2. The method of claim 1, wherein the subject has been diagnosed with cancer.

3. The method of claim 2, wherein the cancer is selected from the group consisting of: esophageal cancer, lung cancer, colon cancer, gastric cancer, and head and neck cancer.

4. The method of claim 1, wherein the one or more microtubule-directed therapies is selected from the group consisting of: taxanes and vinca alkaloids.

5. The method of claim 1, wherein methylation is detected in the promoter region.

6. The method of claim 1, wherein the sample is one or more of blood, blood plasma, serum, cells, a cellular extract, a cellular aspirate, tissues, a tissue sample, or a tissue biopsy.

7. The method of claim 1, wherein the sample is obtained from a subject.

8. The method of claim 1, wherein the method determines the course of treatment.

9. A method for predicting survival in a subject with cancer comprising: detecting nucleic acid methylation of CHFR in one or more samples, wherein detecting nucleic acid methylation identifies survival in a subject.

10. The method of claim 9, wherein the subject underwent previous treatment with a microtubule-directed therapy.

11. A method for identifying a risk of developing cancer in a subject that was treated with a microtubule-directed agent, comprising: detecting nucleic acid methylation of CHFR in one or more samples, wherein detecting nucleic acid methylation identifies a risk of developing cancer in the subject, or
A method for identifying a subject that will respond to one or more microtubule-directed therapies comprising:
extracting nucleic acid from one or more cell or tissue samples;
detecting nucleic acid methylation of the CHFR gene in the sample; and
identifying the nucleic acid methylation state of the CHFR gene,
wherein nucleic acid methylation of CHFR indicates the subject that will respond to one or more microtubule-directed therapies, or
A method for predicting survival in a subject with cancer comprising:
extracting nucleic acid from one or more cell or tissue samples;
detecting nucleic acid methylation of the CHFR gene in the sample; and
identifying the nucleic acid methylation state of the CHFR gene,
wherein nucleic acid methylation of the CHFR gene is indicative of survival in a subject with cancer, or
A method for identifying a risk of developing cancer in a subject that was treated with a microtubule-directed therapy comprising:
extracting nucleic acid from one or more cell or tissue samples;
detecting nucleic acid methylation of the CHFR gene in the sample; and
identifying the nucleic acid methylation state of the CHFR gene,
wherein nucleic acid methylation of the CHFR gene is indicative of a risk of developing cancer in a subject that was treated with a microtubule-directed therapy, or
A method of treating a subject having or at risk for having cancer comprising:
identifying nucleic acid methylation of the CHFR gene, where nucleic acid methylation indicates having or a risk for cancer; and
administering to the subject a therapeutically effective amount of a demethylating agent,
thereby treating a subject having or at risk for having cancer.

12-34. (canceled)

35. The method of claim 34, wherein the method is used in combination with one or more microtubule-directed therapies.

36. The method of claim 35, wherein the one or more microtubule-directed therapies is selected from the group consisting of: taxanes and vinca alkaloids.

37. The method of claim 34, wherein the cancer is selected from the group consisting of: esophageal cancer, lung cancer, colon cancer, gastric cancer, and head and neck cancer.

38-49. (canceled)

50. A kit for identifying the nucleic acid methylation state of a CHFR gene comprising gene specific primers for use in polymerase chain reaction (PCR), and instructions for use, or
A kit for detecting cancer by detecting nucleic acid methylation of a CHFR gene, the kit comprising gene specific primers for use in polymerase chain reaction (PCR), and instructions for use.

51. (canceled)

52. The kit of claim 50, wherein the PCR is methylation specific PCR (MSP).


SEQUENCE LISTING (8 sequences)

SEQ ID NO: 1 - DNA, 3138 bp, Homo sapiens
ctcttgacagcggcggcggcgcagccggttccgggttcggcgcggggcggggatgtgaatcccgatggagcggcccgaggaaggcaagcagtcgccgccgccgcagccctggggacggctcctgcgtctgggcgcggaggagggcgagccgcacgtcctcctgaggaagcgggagtggaccatcgggcggagacgaggttgcgacctttccttccccagcaataaactggtctctggagatcactgtagaattgtagtggatgaaaaatcaggtcaggtgacactggaagataccagcaccagtggaacagtgattaacaagctgaaggttgttaagaagcagacatgccctttacagactggggatgtcatctacttggtgtacaggaagaatgaaccggaacacaacgtggcatacctctatgaatctttaagtgaaaagcaaggcatgacacaagaatcctttgagatggtgccttgctgtgttgcccaggctggtctaaaactcctgggatcaagtgatcctcccaccttggcctcccaaagtattgtgattacagggtctgggggtggtggcatctcccctaaaggaagtggtccctctgtggcaagtgatgaagtctccagctttgcctcagctctcccagacagaaagactgcgtccttttcgtcgttggaaccccaggatcaggaggatttggagcccgagaagaagaaaatgagaggagatggggaccttgacctgaacgggcagttgttggtcgcacaaccgcgtagaaatgcccaaaccgtccacgaggacgtcagagcagcggctgggaagccagacaagatggaggagacgctgacatgcatcatctgccaggacctgctgcacgactgcgtgagtttgcagccctgcatgcacacgttctgcgcggcttgctactcgggctggatggagcgctcgtccctgtgtcctacctgccgctgtcccgtggagcggatctgtaaaaaccacatcctcaacaacctcgtggaagcatacctcatccagcatccagacaagagtcgcagtgaagaagatgtgcaaagtatggatgccaggaataaaatcactcaagacatgctgcagcccaaagtcaggcggtctttttctgatgaagaagggagttcagaggacctgctggagctgtcagacgttgacagtgagtcctcagacattagccagccatacgtcgtgtgccggcagtgtcctgagtacagaaggcaggcggcgcagcctccccactgcccagcacccgagggcgagccaggagccccacaggccctgggggatgcaccccccacgtccgtcagcctgacgacagcagtccaggattacgtgtgccctctgcaaggaagccacgccctgtgcacctgctgcttccagcccatgcccgaccggagagcggagcgcgagcaggacccgcgtgtcgcccctcagcagtgtgcggtctgcctgcagcctttctgccacctgtactggggctgcacccggaccggctgctacggctgcctggccccgttttgtgagctcaacctgggtgacaagtgtctggacggcgtgctgaacaacaacagctacgagtcagacatcctgaagaattacctggcaaccagaggtttgacatggaaaaacatgttgaccgagagcctcgtggctctccagcggggagtgtttctgctgtctgattacagagtcacgggagacaccgttctgtgttactgctgtggcctgcgcagcttccgtgagctgacctatcagtatcagcagaacattcctgcttccgagttgccagtggccgtaacatcccgtcctgactgctactggggccgtaactgccgcactcaggtgaaagctcaccacgccatgaaattcaatcatatctgtgaacagacaaggttcaaaaactaagcatccagaggccctgagcagctttcagcactggaggtgaagagagcgtgtttttaaaatacagaggcaagcacgtcaaggtgttttcacagccccctgagggaagggacgcagggtctccgacaggtgctctggggtgactcttctgtggagctttaccctctgagtgagaccctccccagagccccgggggccgcagcccgccctcctggtgagcgctgggcagggctcgtggtggcatcagcagcagagacgaagcctttctgtaacatgcggccgtcctgccgagaggggcagttttgctcttttgtacattttccgaaactacagttaaagcggaagtctgttttcaggaaaagtttcaagggagaagggcaagtttatcaaaaacattgtttcaggagaagggagcataagtttacagcctacaggacgtacacaatatcctgctgctgggaaaaccacagcattttatctattttttattttaataggtttggtgcttatcttctaataagatttaaatgtcacaaactgtagcacaaataatataatttataatttacaaattgactaaaattgggtatagtatggtatttgaaagaataagcatatgcttctgtttattaaaaaaagaaaccttccaatgtccaaaactgctaaccctcgacgtggccgccaagttagtcgctccttgctaaccggtgagtgaccgcggccccgagcctggggctggacgcaggtcccaggacatgctgctcccttgtgtgagtgaccgcggccccgagcctggggctggacgcaggtcccaggacgtgctgctcccttgtgtgagtgaccacggccccaagcccagggctggaggcaggtcccaggacgcgccgctccctcatgctgcccgggcccttcctccaagaccctacagagcctgaggggcaccttggcttccgcctgtgctagctttgccatgtcatctggaataatacttgaaattttgattcttggaaaaaaaagtttcttatcttttgttgaaatcacctgttatccttgtttgtaaactgataacttttttgcttcttctcaggaatacagttttcaactgttgtcttgctcttgatagaaactgagaagcagcaatctgtatttgtggaggaaagtcctctcttttgcatattctaataaatgagccgcgtttgctcctc

SEQ ID NO: 2 - protein, 623 aa, Homo sapiens
MERPEEGKQSPPPQPWGRLLRLGAEEGEPHVLLRKREWTIGRRRGCDLSFPSNKLVSGDHCRIVVDEKSGQVTLEDTSTSGTVINKLKVVKKQTCPLQTGDVIYLVYRKNEPEHNVAYLYESLSEKQGMTQESFEMVPCCVAQAGLKLLGSSDPPTLASQSIVITGSGGGGISPKGSGPSVASDEVSSFASALPDRKTASFSSLEPQDQEDLEPEKKKMRGDGDLDLNGQLLVAQPRRNAQTVHEDVRAAAGKPDKMEETLTCIICQDLLHDCVSLQPCMHTFCAACYSGWMERSSLCPTCRCPVERICKNHILNNLVEAYLIQHPDKSRSEEDVQSMDARNKITQDMLQPKVRRSFSDEEGSSEDLLELSDVDSESSDISQPYVVCRQCPEYRRQAAQPPHCPAPEGEPGAPQALGDAPPTSVSLTTAVQDYVCPLQGSHALCTCCFQPMPDRRAEREQDPRVAPQQCAVCLQPFCHLYWGCTRTGCYGCLAPFCELNLGDKCLDGVLNNNSYESDILKNYLATRGLTWKNMLTESLVALQRGVFLLSDYRVTGDTVLCYCCGLRSFRELTYQYQQNIPASELPVAVTSRPDCYWGRNCRTQVKAHHAMKFNHICEQTRFKN

SEQ ID NO: 3 - DNA, 25 bp, Artificial Sequence
  Description of Artificial Sequence Synthetic primer
gttattttcgtgattcgtaggcgac

SEQ ID NO: 4 - DNA, 21 bp, Artificial Sequence
  Description of Artificial Sequence Synthetic primer
cgaaaccgaaaataacccgcg

SEQ ID NO: 5 - DNA, 26 bp, Artificial Sequence
  Description of Artificial Sequence Synthetic primer
tagggagtatataggttggggaagtt

SEQ ID NO: 6 - DNA, 27 bp, Artificial Sequence
  Description of Artificial Sequence Synthetic primer
aacacacaataacaaacacaaattcac

SEQ ID NO: 7 - DNA, 51 bp, Artificial Sequence
  Description of Artificial Sequence Synthetic primer
cgacatgcgaagtcgtttggttaggattaaagatggtcgagcggcatgtcg

SEQ ID NO: 8 - DNA, 42 bp, Artificial Sequence
  Description of Artificial Sequence Synthetic primer
cgactgcgtgtggggtggtgatggaggaggtttaggcagtcg